(19) United States
(12) Patent Application Publication
(10) Pub. No.: US 2026/0294812 A1
(43) Pub. Date: Oct. 1, 2026

(54) CANNABIDIOL FORMULATIONS

(71) Applicant: DSM IP Assets B.V., 6221 BE Maastricht (NL)

(72) Inventors: Igor Bendik, Kaiseraugst (CH); Felix Franz ROOS, Kaiseraugst (CH); Claudia Mareike BECK, Kaiseraugst (CH); Christiane Schweiggert, Kaiseraugst (CH); Bernd Mussler, Kaiseraugst (CH); Deanna Lynn McCarthy, Spring, TX (US)

(21) Appl. No.: 19/302,913

(22) Filed: Aug. 18, 2025

Related U.S. Application Data
(60) Provisional application No. 63/781,857, filed on Apr. 1, 2025.
(60) Provisional application No. 63/781,864, filed on Apr. 1, 2025.
(60) Provisional application No. 63/781,866, filed on Apr. 1, 2025.

(30) Foreign Application Priority Data
May 7, 2025 (EP) 25174807.5
May 7, 2025 (EP) 25174808.3
May 7, 2025 (EP) 25174809.1

(51) Int. Cl. A61K 9/16; A61K 9/48; A61K 31/00

(52) U.S. Cl. CPC A61K 9/1658; A61K 9/1617; A61K 9/1647; A61K 9/1652; A61K 9/1694; A61K 9/4825; A61K 9/4858; A61K 9/4866; A61K 31/658

(57) ABSTRACT

The present invention relates to compositions comprising cannabidiol having improved pharmacokinetics, and therapeutic uses thereof.

### CROSS REFERENCE TO RELATED APPLICATIONS

This application claims priority to U.S. Provisional Applications Nos. 63/781,857, filed 1 Apr. 2025, 63/781,864, filed 1 Apr. 2025, and 63/781,866, filed 1 Apr. 2025, and European Patent Applications Nos. EP25174807.5, filed 7 May 2025, EP25174808.3, filed 7 May 2025, and EP25174809.1, filed 7 May 2025, each of the foregoing of which are incorporated herein by reference in their entireties to the full extent permitted by law.

### TECHNICAL FIELD

The present invention relates to compositions comprising cannabidiol having improved pharmacokinetics, and therapeutic uses thereof.

### BACKGROUND

Cannabidiol (CBD) and delta-9-tetrahydrocannabinol (Δ9-THC) are two major compounds found in the Cannabis sativa plant, and they interact with the body differently. While Δ9-THC has a high affinity for cannabinoid receptor types 1 and 2 (G protein-coupled receptors found primarily on neurotransmitters), CBD has a low affinity and lack of function activity at these receptors which may explain its non-psychoactive effects.

While research is still ongoing, several proven health benefits have been identified for CBD. CBD has been shown to have analgesic (pain-relieving) properties. It interacts with the endocannabinoid system, which regulates pain perception. Studies have demonstrated its potential in reducing chronic pain associated with conditions like arthritis, multiple sclerosis, and neuropathic pain. CBD has also shown promising results in reducing anxiety, particularly in people with generalized anxiety disorder (GAD), social anxiety disorder (SAD), and post-traumatic stress disorder (PTSD). CBD's anxiolytic effects are thought to result from its interaction with serotonin receptors, influencing mood and stress regulation. Further, CBD has neuroprotective properties and has been investigated for its potential in preventing neurodegenerative diseases like Alzheimer's and Parkinson's. It appears to reduce oxidative stress and inflammation in the brain, which may protect against neuronal damage. CBD is well-known for its anti-inflammatory properties, which make it a candidate for managing autoimmune conditions, such as rheumatoid arthritis and Crohn's disease. Its ability to modulate the immune system is thought to be through interactions with the endocannabinoid system. CBD has also shown promise in improving sleep quality, particularly in individuals with insomnia or those suffering from anxiety-related sleep disturbances. Research suggests CBD may have a role in regulating sleep cycles by interacting with receptors that influence the sleep-wake cycle. CBD is further being investigated for potential health benefits for diseases of the central nervous system (multiple sclerosis, amyotrophic lateral sclerosis, Parkinson's disease, Huntington's disease, autism, schizophrenia), opioid use disorders, Tourette's syndrome, glaucoma, IBD, IBS, as well as cancer and cancer related illnesses (including gliomas, glioblastomas, colorectal cancer, pancreatic cancer, lung cancer, breast cancer). The physicochemical properties of CBD make its clinical development challenging. CBD is a lipophilic compound which has poor solubility, variable pharmacokinetic (PK) profiles, poor oral bioavailability, and food interactions.

The bioavailability of CBD after oral dosing under fasting conditions is only about 6% and increases about 4-fold when it is co-administered with a high-fat meal (Perucca E, Bialer M, CNS Drugs. 2020; 34:795-800) (“A formulation with improved biopharmaceutical properties could increase the extent of CBD absorption about fourfold (i.e., to the level achieved with the currently available formulations co-administered with a high-fat meal) and minimize the influence of food effects on CBD bioavailability.”). CBD is rapidly metabolized by the liver to 7-hydroxycannabidiol (7-OH-CBD) and then to cannabidiol-7-oic acid (7-COOH)-CBD) (Abbotts K S S, Ewell T R, Butterklee H M, et al. Nutrients. 2022; 14(10)).

In 2018, the first CBD containing drug, Epidiolex®, was approved in the United States by the US Food and Drug Administration (FDA). Epidiolex was found to be effective in the treatment of certain types of epilepsy, like the Dravet Syndrome (DS), Lennox-Gaustaut Syndrome (LGS), and tuberous sclerosis complex (TSC). In Europe, it is marketed under the tradename Epidyolex®. Epidiolex (Epidyolex) is a purified form of CBD delivered as an alcoholic sesame oil solution. It is administered in a liquid form.

Administration of CBD in liquid/oil form is not ideal for patients as it is neither convenient nor precise. A solid format (e.g., a powder in the form of a capsule or tablet) is considered more patient-friendly and enables more precise dosing, preferably with a high CBD loading to simplify administration and enhance compliance.

Epidiolex prescribing information highlights the fact that CBD should be taken consistently with or without food to reduce pharmacokinetic variability (Epidiolex. Full Prescribing Information, item 2.4: “Food may affect EPIDIOLEX levels. Consistent dosing of EPIDIOLEX with respect to meals is recommended to reduce variability in cannabidiol plasma exposure.” https://www.accessdata.fda.gov/drugsatfda_docs/label/2024/210365s021lbl.pdf). Taking the medication inconsistently with respect to mealtimes could lead to changes over time in disease control and clinical tolerability. From a clinical viewpoint, reducing inter- and intra-subject pharmacokinetic variability would therefore be expected to result in a more consistent clinical response over time. A CBD formulation with less pharmacokinetic variability would help reduce the susceptibility to food effects and/or minimize the effect of food on CBD exposure.

In view of the above, there is a need in the art for oral CBD formulations with improved pharmacokinetic profiles. For example, a CBD formulation which reaches the maximum concentration of CBD quicker would result in a quicker onset of action of the CBD and accelerate and increase its effect. Further, a CBD formulation with a higher bioavailability would enhance treatment effectiveness and improve patient experience, in particular in fasted individuals in which the bioavailability of CBD is known to be generally lower than in fed state individuals.

### SUMMARY

Disclosed herein are compositions comprising cannabidiol having improved pharmacokinetics, and therapeutic uses thereof. In certain embodiments, the composition comprises a therapeutically effective amount of cannabidiol or a pharmaceutically acceptable salt thereof, Vitamin E (tocopherol) and/or one or more derivatives of Vitamin E. In one embodiment, the composition comprises:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of tocopherol polyethylene glycol succinate; and
(iv) one or more excipients or additives in an amount to make the composition total 100 wt.-%.

The pharmaceutical composition may be formulated into a tablet (e.g., orally dispersible tablet, chewable tablet, film coated tablet, immediate release tablet, extended or sustained release tablet and similar), a capsule, an orally dispersible film, a gummy, or a powder. In another embodiment, the composition is a spray dried powder that is formulated into such dosage forms.

In certain embodiments, the pharmaceutical compositions described herein, when administered to a patient in need thereof, provide therapeutically effective pharmacokinetic levels regardless of whether the patient is in a fed or fasted state. For example, the oral pharmaceutical composition according to the present invention provides a higher Cₘₐₓ and a significantly earlier Tₘₐₓ of CBD in plasma, when compared to the existing liquid formulation Epidyolex, when administered at equal CBD dosage level and when administered in a fed state. Such higher Cₘₐₓ and a significantly earlier Tₘₐₓ of CBD in plasma occurs, in another embodiment, when the inventive composition is administered in a fasted state. Further, an oral pharmaceutical composition comprising cannabidiol, Vitamin E and TPGS results in a 15% higher plasma AUC₀₋₂₄ of CBD and a 49% higher plasma AUC₀₋₂₄ of 7-OH CBD (p<0.001), when compared to the existing liquid formulation Epidyolex, when administered at equal CBD dosage level and when administered in a fasted state.

In another embodiment, an oral pharmaceutical composition comprising the spray dried CBD powder according to the present invention has improved pharmacokinetic properties with respect to variability when compared to the existing liquid formulation Epidyolex when administered at equal CBD dosage level and regardless of proximity to meals. As shown in the study described herein, the coefficient of variation (CV) for AUC₀₋₂₄ and Cₘₐₓ was substantially lower for the inventive composition, both in fasted and fed state.

The disclosed methods generally comprise the use of the pharmaceutical compositions in treating patients who would benefit from treatment with cannabidiol. Such uses may be in the prevention or treatment of a patient suffering from a disease or condition selected from: chronic pain; neuropathic pain; post-operative pain (e.g., dental procedure or oral surgery), anxiety, including generalized anxiety disorder (GAD), social anxiety disorder (SAD), and post-traumatic stress disorder (PTSD); neurodegenerative diseases, including Alzheimer's and Parkinson's; autoimmune conditions, including rheumatoid arthritis and Crohn's disease; sleep disorders, including insomnia and anxiety-related sleep disturbances; diseases of the central nervous system, including multiple sclerosis, amyotrophic lateral sclerosis, Parkinson's disease, Huntington's disease, autism, and schizophrenia; opioid use disorders; Tourette's syndrome; glaucoma; IBD; IBS; and cancer and cancer related illnesses, including gliomas, glioblastomas, colorectal cancer, pancreatic cancer, lung cancer, and breast cancer.

Additional embodiments of the present compositions, methods of treatment, processes and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE DRAWINGS

The summary, as well as the following detailed description, is further understood when read in conjunction with the appended drawings. For the purpose of illustrating the disclosed compositions and methods, there are shown in the drawings exemplary embodiments of the same; however, the compositions and methods are not limited to the specific embodiments disclosed. In the drawings:

FIG. 1 shows the arithmetic mean CBD levels in plasma in fasted patients following ingestion of 400 mg CBD as either test product (TP, solid line with squares) or reference product (RP, Epidyolex, dashed line with diamonds). The standard error of the mean (SEM) is shown as error bars. With both forms, the CBD plasma levels increase rapidly within 2 hours, reach a peak after 3-4 hours, and then decrease exponentially. However, the curve for the TP increases slightly faster than for Epidyolex, reaches a higher maximum (Cₘₐₓ), and the area under curve (AUC) is also higher than for Epidyolex.

FIG. 2 shows the mean curves with standard error of the mean (SEM) as error bar for 7-OH-CBD, a biologically active, immediate downstream metabolite of CBD. The upper curve is for the test product (TP), the lower curve is for the reference product (RP, Epidyolex). In fasted patients the test product achieves an area under the curve (AUC) for CBD which is 49% higher than with Epidyolex (p<0.001).

FIG. 3 shows the arithmetic mean CBD levels in plasma following ingestion of 400 mg CBD as either test product (TP, solid line with squares) or reference product (RP, Epidyolex, dashed line with diamonds) in fed patients. The standard error of the mean (SEM) is shown as error bars. With the test product, CBD plasma levels increased steeply after 2 hours and reached their maximum (Cₘₐₓ) around 5-6 hours on average. With Epidyolex, the increase was much slower and variable, reaching the maximum (Cₘₐₓ) after 8-12 hours.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable. The disclosure of ranges is intended as a continuous range including every value between the minimum and maximum values.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any sub-combination.

### A. DEFINITIONS

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

The term “about,” as used herein, is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%. When values are expressed as approximations, by use of the antecedent “about,” it will be understood that the particular value forms another embodiment. Reference to a particular numerical value includes at least that particular value, unless the context clearly dictates otherwise.

The term “bioequivalent,” as used herein, denotes a scientific basis on which two or more pharmaceutical products, compositions or methods containing same active ingredient are compared with one another. “Bioequivalence” means the absence of a significant difference in the rate and extent to which the active agent in pharmaceutical equivalents or pharmaceutical alternatives becomes available at the site of action when administered in an appropriately designed study. Bioequivalence can be determined by an in vivo study comparing a pharmacokinetic parameter for the two compositions. Parameters often used in bioequivalence studies are Tₘₐₓ, Cₘₐₓ, AUC_{0-inf}, AUC₀₋ₜ. In the present context, substantial bioequivalence of two compositions or products is established by 90% confidence intervals (CI) of between 0.80 and 1.25 for AUC and Cₘₐₓ.

The term “consisting essentially of” as used in the context of the invention means that the addition of the wt-% of the listed ingredients add up to 100 wt.-%. However, it cannot be excluded that small amounts of impurities may be present such as, e.g., in amounts of less than 5 wt.-%, preferably less than 3 wt.-% which are introduced via the respective raw materials, processes used or formed, e.g. degradation products.

In the context of the present invention, the term “crystalline” is understood to mean a coherent or non-coherent part of one or more components with homogeneous physical properties, in particular a homogeneous melting range and a long-range order. It is well understood that crystals are solid, i.e. in a solid state of aggregation.

In the context of the present invention, the term “non-crystalline” is understood to mean a cohesive or non-cohesive part of one or more components with homogeneous physical properties, in particular without any homogeneous melting range and long-range order.

The term “effective amount” or “therapeutically effective amount” refers to an amount necessary to obtain the desired physiological effect and may depend on the condition to be treated. The physiological effect may be achieved by one application dose or by repeated applications. The dosage administered may, of course, vary depending upon known factors, such as the mode and route of administration; the age, health and weight of the recipient; the nature and extent of the symptoms; the kind of concurrent treatment; the frequency of treatment; and the effect desired and can be adjusted by a person skilled in the art.

In the context of the present invention, the term “long range order” is understood to mean a regular and periodic spacing of molecules or atoms (here of the at least one cannabinoid) in a crystalline solid. Accordingly, the exact position of a few molecules or atoms can advantageously be used to determine the position of all molecules or atoms in a crystalline solid.

As used herein, the terms “fasted,” “fasted state,” “in a fasted state,” “fasted condition,” “fasting condition” and “without food” are used interchangeably and mean, in general, the state/condition of not having consumed food for at least 3 hours, preferably at least 5 hours, most preferably at least 7 hours (such as at least 10 hours, e.g. about 10.5 hours) prior to the administration of the pharmaceutical composition of the present invention, and for at least 1 hour, preferably at least 2 hours, most preferably at least 3 hours (such as at least 4 hours) after the administration of the pharmaceutical composition of the present invention. Preferably, the term “fasted” and the like refer to a state of not having consumed food for a time period of at least about 3 to about 15 hours, preferably 5 to 12 hours, most preferably 8 to 12 hours prior to the administration of the pharmaceutical composition of the present invention and at least about 1 to 5 hours, preferably 2 to 4 hours, most preferably about 4 hours after the administration of the pharmaceutical composition of the present invention.

As used herein, the terms “fed,” “fed state,” “in a fed state,” “with food” are used interchangeably and mean, in general, the state/condition of having consumed food prior to, during and/or after the administration of the pharmaceutical composition of the present invention. Preferably, the term “in a fed state” and the like refer to a state of (i) having consumed food at least 1 hours, preferably at least 45 min, most preferably at least 30 min prior to the administration of the pharmaceutical composition of the present invention, (ii) consuming food during the administration of the pharmaceutical composition of the present invention, and/or (iii) consuming food within a period up to about 2 hour, preferably up to 1 hours, most preferably up to 30 min after the administration of the pharmaceutical composition of the present invention. Preferably, the term “in a fed state” and the like refer to the state of having consumed food for a time period of between from at least about 1 to 15 hours, preferably 5 to 12 hours, most preferably 8 to 12 hours prior to the administration of the pharmaceutical composition of the present invention and at least about 1 to 5 hours, preferably 2 to 4 hours most preferably about 4 hours after the administration of the pharmaceutical composition of the present invention. Preferably, the food is a solid food with sufficient bulk and fat content that is not rapidly dissolved and absorbed in the stomach. More preferably, the food is a meal, such as breakfast, lunch or dinner. Preferably, the term “in the fed state” refers to an administration within 30 minutes before a meal until 2 hours after a meal, preferably during a meal or shortly after a meal such as up to 1 hour after a meal, preferably up to 30 min after a meal. In an even more preferred embodiment, the meal is a high-fat, high calorie meal. Most preferably, such high fat/calorie meal provides at least 800 to 1000 kcal.

As used herein, the term “Particle Size Distribution (PSD)” means the cumulative volume size distribution of equivalent spherical diameters as determined by laser diffraction in Malvern Master Sizer 2000 equipment or its equivalent.

The term “pharmaceutically acceptable” means that which is useful in preparing a pharmaceutical composition that is generally non-toxic and is not biologically undesirable. It includes that which is acceptable for veterinary use and/or human pharmaceutical use.

The term “pharmaceutical composition” is intended to encompass a drug product including the active ingredient(s), pharmaceutically acceptable excipients such as diluents, binders, glidants, disintegrants, pharmaceutically acceptable salts and/or the like that make up the carrier, as well as any product which results, directly or indirectly, from combination, complexation or aggregation of any two or more of the ingredients. Accordingly, in one example, the pharmaceutical compositions encompass any composition made by admixing the spray dried powder according to the present disclosure, optionally additional active ingredient(s), and pharmaceutically acceptable excipients.

### B. OVERVIEW OF DETAILED DESCRIPTION

Disclosed herein are cannabidiol-containing pharmaceutical compositions having advantageous pharmaceutic, pharmacokinetic and pharmacodynamic properties. In a broad aspect, the composition comprises a therapeutically effective amount of cannabidiol or a pharmaceutically acceptable salt thereof, Vitamin E (tocopherol) and/or one or more derivatives of Vitamin E. In one embodiment, the composition comprises:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of tocopherol polyethylene glycol succinate; and
(iv) one or more excipients or additives in an amount to make the composition total 100 wt.-%.

The pharmaceutical composition may be formulated into a tablet (e.g., orally dispersible tablet, chewable tablet, sublingual tablet, film coated tablet, immediate release tablet, extended or sustained release tablet and similar), a capsule, an orally dispersible film, a gummy, or a powder. In another embodiment, the composition is a spray dried powder that is formulated into such dosage forms.

The disclosed methods generally comprise the use of the pharmaceutical compositions in treating patients who would benefit from treatment with cannabidiol. This includes, without limitation, patients with seizure disorders, chronic pain (e.g., neuropathic pain), anxiety, post-operative pain (e.g., dental procedure or oral surgery), depression, sleep disorders, inflammatory diseases, neurodegenerative diseases, opioid use disorder, post-traumatic stress disorder (PTSD), Alzheimer's disease, and other cognitive illnesses.

### C. COMPOSITIONS AND DOSAGE FORMS

The pharmaceutical compositions described herein generally comprise a therapeutically effective amount of cannabidiol or a pharmaceutically acceptable salt thereof, Vitamin E (tocopherol) and/or one or more derivatives of Vitamin E. Such derivatives include, but are not limited to, tocopheryl acetate, tocopheryl succinate, tocopheryl nicotinate, tocopherol linoleate, tocopherol palmitate, tocopherol phosphate, tocopherol retinoate, and tocopheryl polyethylene glycol succinate (TPGS) (collectively, “Vitamin E Derivative(s)”).

In one embodiment, the composition comprises:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of a Vitamin E Derivative; and
(iv) one or more excipients or additives in an amount to make the composition total 100 wt.-%.

In another embodiment, the Vitamin E Derivative is selected from the group consisting of tocopheryl acetate, tocopheryl succinate, tocopheryl nicotinate, tocopherol linoleate, tocopherol palmitate, tocopherol phosphate, tocopherol retinoate, and tocopheryl polyethylene glycol succinate (TPGS) or mixtures of any of the foregoing. In one example, the Vitamin E Derivative is TPGS in an amount of about 1 to 10 wt.-% based on the total weight of the composition. The pharmaceutical composition may further comprise one or more of:
(a) an antioxidant in an amount of about 1 to 10% based on the total weight of the composition;
(b) a glidant in an amount of about 1 to 5% based on the total weight of the composition; and
(c) water in an amount of about 1 to 5% based on the total weight of the composition.

In certain embodiments, the excipient or additive in the above compositions may comprise (a) a sugar alcohol such as mannitol, sorbitol, xylitol, erythritol, maltitol, isomalt, and/or lactitol; (b) a maltodextrin (such as maltodextrin Glucidex®), and/or tapioca starch; (c) microcrystalline cellulose, crospovidone (polyvinylpolypyrrolidone), croscarmellose sodium, carboxy-methylcellulose (CMC), CMC sodium, hypromellose, and/or sodium starch glycolate; (d) gelatine (e.g., porcine gelatine 140 bloom); and/or (e) a coating if the dosage form is a tablet or other form that may benefit from a coating. The antioxidant in the above composition may comprise an ascorbic acid, such as L-ascorbic acid, butylated hydroxytoluene (BHT), butylated hydroxyanisole (BHA), and/or a sulfite. The glidant in the above composition may comprise silicon dioxide, talc, glycerol monostearate, magnesium stearate, and/or a precipitated silica.

More generally, suitable pharmaceutically acceptable excipients, carriers or additives to be used in the pharmaceutical composition according to the present invention encompass diluents, like lactose, starch, microcrystalline cellulose, sorbitol, mannitol, dibasic calcium phosphate dihydrate, calcium sulfate dihydrate, sucrose-based diluents and mixtures thereof; binders, like acacia, cellulose derivatives, gelatine, glucose, polyvinylpyrollidone, starch, sucrose, sorbitol, tragacanth, sodium alginate and mixtures thereof; disintegrants, like microcrystalline cellulose and cellulose derivatives, starch and its derivatives, alginic acid and its derivatives, ion-exchange resins, cross-linked sodium carboxymethyl cellulose, sodium starch glycolate, cross-linked polyvinylpyrrolidone and formaldehyde-casein; as well as lubricants, anti-adherents and glidants, like magnesium-, calcium- and sodium stearates, stearic acid, hydrogenated castor oil, talc, water, polyethylene glycol, sodium lauryl sulfate, magnesium lauryl sulfate and silica. Particularly preferred is the use of silica and/or mannitol.

The present disclosure further encompasses pharmaceutically acceptable salts of cannabidiol, such as sodium, potassium, lithium, tetrabutylammonium and choline hydroxide. In one embodiment, acid addition salts are prepared from the free base forms using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. The following list of organic and inorganic acids is not meant to be exhaustive but merely illustrative as person of ordinary skill in the art would appreciate that other acids may be used to create pharmaceutically acceptable salts of cannabidiol and prodrugs of cannabidiol.

Applicant's additional cannabinoid formulations are described in PCT/EP2024/056835 (WO2024/189150), incorporated herein by reference.

In certain embodiments, the composition is a spray dried powder. The amount of the spray dried powder of the present invention with all the preferences and definitions given herein in the pharmaceutical composition according to the present invention is preferably at least 75 wt.-%, more preferably at least 80 wt.-%, most preferably at least 85 wt.-%, based on the total weight of the pharmaceutical composition.

In one embodiment, the present invention relates to a spray dried powder (A) comprising:
(i) 30-45 wt.-%, based on the total weight of the spray dried powder, of cannabidiol,
(ii) 5 to 15 wt.-%, based on the total weight of the spray dried powder, of Vitamin E,
(iii) 2.5 to 7.5 wt.-%, based on the total weight of the spray dried powder, of tocopherol polyethylene glycol succinate,
(iv) 25 to 40 wt.-%, based on the total weight of the spray dried powder, of gelatine,
(v) 4 to 10 wt.-%, based on the total weight of the spray dried powder, of hydrolysed starch having a DE value selected in the range of 8 to 38,
(vi) 0.5 to 3 wt. %, based on the total weight of the spray dried powder, of an additional antioxidant (other than Vitamin E), preferably ascorbic acid or a pharmaceutically acceptable salt thereof, and
(vii) less than 5 wt.-%, based on the total weight of the spray dried powder, of water.

In a preferred embodiment, the present invention relates to a spray dried powder (A-I), which is a spray dried powder (A) comprising cannabidiol hydroxyquinone (CBDHQ) in an amount of up to 0.5 wt.-%, preferably up to 0.2 wt.-%, most preferably up to 0.1 wt.-% (as determined via HPLC-DAD), based on the total with of the spray dried powder. Suitable ranges of CBDHQ in the spray dried powders according to the present invention are selected in the range from 0.0001 wt.-% to 0.5 wt.-%, from 0.001 wt.-% to 0.5 wt.-%, from 0.01 wt.-% to 0.5 wt.-%, from 0.025 wt.-% to 0.5 wt.-%, from 0.0001 wt.-% to 0.2 wt.-%, from 0.001 wt.-% to 0.2 wt.-%, from 0.01 wt.-% to 0.2 wt.-%, from 0.025 wt.-% to 0.2 wt.-%, based on the total weight of the respective spray dried powder.

In an even more preferred embodiment, the present invention relates to a spray dried powder (A-II) which is a spray dried powder (A) or (A-I) having a particle size distribution as measured by laser diffraction (Malvern) characterized by a D10>1 μm, a D50 in the range from 1-200 μm and a D90<500 μm, preferably by a D10>5 μm, a D50 selected in the range from 10-150 μm and a D90<400 μm, most preferably by a D10>8 μm, a D50 selected in the range from 20-100 μm and a D90<300 μm.

Particularly preferred in all embodiments of the present invention is a spray dried powder (A-III), which is a spray dried powder A-(I) or (A-II) comprising CBDHQ in an amount of at most 0.2 wt.-% (as determined via HPLC-DAD) and having a particle size distribution as measured by laser diffraction (Malvern) of D10>8 μm, D50 selected in the range from 20-100 μm and D90<300 μm.

In a second aspect, the present invention relates to a composition comprising the spray dried powder (A), (A-I), (A-II) or (A-III). The composition may be a food, a dietary supplement, a nutraceutical, or a pharmaceutical composition. In a preferred aspect, the composition is a pharmaceutical composition comprising the spray dried powder (A), (A-I), (A-II) or (A-III) and a pharmaceutically acceptable carrier.

Preferably, the composition, including the pharmaceutical composition, is a solid composition intended for oral intake, i.e., a solid oral dosage form.

Preferably, in all embodiments of the present invention, the pharmaceutical composition according to the present invention comprises at least 20 wt.-%, more preferably at least 25 wt.-%, most preferably at least 30 wt.-%, of cannabidiol, based on the total weight of the pharmaceutical composition.

Preferably, in all embodiments of the present invention, the pharmaceutical composition according to the present invention comprises less than 3 wt.-% of water, more preferably less than 2 wt.-% of water, based on the total weight of the pharmaceutical composition. Most preferably water is contained in the range from 1-2 wt.-%, based on the total weight of the pharmaceutical composition.

Preferably, in all embodiments of the present invention, the spray dried powders according to the present invention consist essentially of the components (i) to (vii).

The important characteristics of the PSD are D90 (also often referred to as D (v, 0.9)), which is the size, in microns, below which 90% of the particles by volume are found, the D50 (also often referred to as D (v, 0.5)), which is the size, in microns, below which 50% of the particles by volume are found and the D10 (also often referred to as D (v, 0.1)), which is the size, in microns, below which 10% of the particles by volume are found. Thus, a D90 of less than 500 microns means that 90 volume-percent of the particles in the spray dried powder according to the present invention have a diameter of less than 500 microns.

Vitamin E is a group of eight fat soluble compounds that may comprise four tocopherols ((a)-tocopherol, (b)-tocopherol, (g)-tocopherol and (d)-tocopherol) and four tocotrienols ((a)-tocotrienol, (b)-tocotrienol, (g)-tocotrienol and (d)-tocotrienol). Also, mixtures of these compounds can be used such as (all-rac)-α-tocopherol. In the context of the present invention (all-rac)-α-tocopherol is preferred in all embodiments, which is e.g. commercially available at DSM Nutritional Products Ltd as dl-α-Tocopherol or all-rac-α-Tocopherol.

Tocopherol polyethylene glycol succinate (TPGS), also known as yocofersolan (INN) or tocophersolan, is a synthetic water-soluble version of Vitamin and is e.g. commercially available as Vitamin E TPGS from Sigma-aldrich or Merck. The IUPAC name of tocopherol polyethylene glycol succinate is α-Hydro-w-{[4-oxo-4-({(2R)-2,5,7,8-tetramethyl-2-[(4R,8R)-4,8,12-trimethyltridecyl]-3,4-dihydro-2H-1-benzopyran-6-yl}oxy)butanoyl]oxy}poly(oxyethylene). The CAS number is 9002-96-4.

It is particularly preferred that the spray dried powder according to the present invention contains no further oil next to the Vitamin E, i.e. Vitamin E is the sole oil used in the preparation process to solubilize the cannabidiol present in the spray dried powders according to the present invention.

Tocopherol polyethylene glycol succinate is the compound of the following formula:

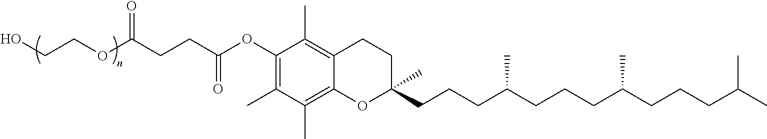

The gelatine to be used in the spray dried powder according to the present invention can be derived from bovine, fish, pork, poultry, and plant proteins as well as mixtures thereof. Preferably, in all embodiments of the present invention pork skin gelatine (porcine skin gelatine) is used.

Preferably, in all embodiments of the present invention, the hydrolysed starch exhibits a DE (Dextrose Equivalent) value selected in the range from 12-29, more preferably in the range from 17-21. The starch can be derived from wheat, maize, potato. Preferably, the hydrolysed starch used according to the present invention is derived from wheat. Such hydrolysed starch is commercially available and sold as dried solid glucose sirup or maltodextrin (CAS 9050-36-6), the use of maltodextrin being particularly preferred according to the present invention.

The ascorbic acid used in the present invention preferably is commercially available ascorbic acid used as a component in compositions in the field of pharmaceuticals, quasi-drugs, foods and dietary supplements. More preferably L-ascorbic acid is used. Ascorbic acid salts can also be used. Here, the ascorbic acid salts are pharmaceutically acceptable salts. There are no particular restrictions, and examples include both salts with an organic base (for example, salts with tertiary amines such as trimethylamine salts, triethylamine salts, monoethanolamine salts, triethanolamine salts and pyridine salts, as well as basic ammonium salts such as arginine), and salts with an inorganic base (for example, alkali metal salts such as ammonium salts, sodium salts and potassium salts, alkaline earth metal salts such as calcium salts and magnesium salts, and aluminum salts). Preferred ascorbic acid salts are sodium salts and potassium salts. A specific example includes sodium ascorbate. Most preferably, in all embodiments of the present invention L-ascorbic acid is used.

The term “less than 5 wt.-% of water” refers to any amount selected in the range from 0 to <5 wt.-%, such as from 0 to 4.999 wt.-%, based on the total weight of the spray dried powder. Particular advantageous spray dried powders according to the present invention comprise from 1 to <5 wt.-%, from 2 to <5 wt.-% or from 2.5 to <5 wt.-% of water, based on the total weight of the spray dried powder. Further suitable ranges are from 0.5 to 4 wt.-%, 1 to 4 wt.-%, 0.5 to 3 wt.-%, 1 to 3 wt.-% and 2 to 3 wt.-%.

In one particular preferred embodiment of the present invention, the spray dried powder (A), (A-I), (A-II) or (A-III) is a spray dried powder (A′), (A-I′), (A-II′) or (A-III′) consisting essentially of
(i) 35-42 wt.-%, based on the total weight of the spray dried powder, of cannabidiol,
(ii) 5 to 15 wt.-%, based on the total weight of the spray dried powder, of (all-rac)-α-tocopherol,
(iii) 3 to 7.5 wt.-%, based on the total weight of the spray dried powder, of tocopherol polyethylene glycol succinate,
(iv) 25 to 40 wt.-%, based on the total weight of the spray dried powder, of gelatine,
(v) 4 to 10 wt.-%, based on the total weight of the spray dried powder, of hydrolysed starch having a DE value selected in the range from 12-29,
(vi) 0.5 to 3 wt. %, based on the total weight of the spray dried powder, of ascorbic acid or a pharmaceutically acceptable salt thereof, and
(vii) 1-4 wt.-%, based on the total weight of the spray dried powder, of water.

In another particular embodiment of the present invention, the spray dried powder (A), A(-I), (A-II) or (A-III) is a spray dried powder (A″), (A-I″), (A-II″) or ((A-III″) consisting essentially of
(i) 35-42 wt.-%, based on the total weight of the spray dried powder, of cannabidiol,
(ii) 5 to 15 wt.-%, based on the total weight of the spray dried powder, of (all-rac)-α-tocopherol,
(iii) 3 to 7.5 wt.-%, based on the total weight of the spray dried powder, of tocopherol polyethylene glycol succinate,
(iv) 25 to 40 wt.-%, based on the total weight of the spray dried powder, of pork gelatine having a bloom value selected in the range from 100 to 200,
(v) 4 to 10 wt.-%, based on the total weight of the spray dried powder, of hydrolysed starch having a DE value selected in the range of 17-21,
(vi) 0.5 to 3 wt. %, based on the total weight of the spray dried powder, of L-ascorbic acid, and
(vii) 1-3 wt.-%, based on the total weight of the spray dried powder, of water.

Preferably, in all embodiments of the present invention, the ratio (w/w) of cannabidiol to Vitamin E in the spray dried powders according to the present invention is selected in the range from 2:1 to 4:1, more preferably from 3:1 to 4:1. Most preferably the ratio is about 4.

Preferably, in all embodiments of the present invention, the ratio (w/w) of the gelatine to tocopherol polyethylene glycol succinate in the spray dried powders according to the present invention is selected in the range of 10:1 to 3:1, preferably 8:1 to 5:1, most preferably 7:1 to 6:1.

Preferably, in all embodiments of the present invention, the emulsion inner droplet size (i.e. the droplets comprising the solubilized cannabidiol) in the spray dried powders according to the present invention is less than 150 nm as measured by laser diffraction (Malvern).

Without being bound to theory, the crystallinity of the cannabidiol in the spray dried powders according to the present invention is less than 2.0% @ release of the spray dried powder, i.e. after production and does not exceed 7.0% upon shelf-life (storage).

Preferably, in all embodiments of the present invention, at least 90 wt-%, preferably at least 91 wt.-%, more preferably at least 92 wt.-%, most preferably at least 93 wt.-%, based on the total weight of the cannabidiol, of the cannabidiol in the spray dried powders is in a non-crystalline (amorphous) form.

In all embodiments of the present invention, most advantageously, the non-crystalline cannabidiol comprised in the spray dried powders according to the present invention is liquid, i.e. is present in a liquid (e.g. dissolved) state of aggregation dissolved in the Vitamin E. Even more preferably, the non-crystalline cannabinoid is not or not substantially amorphous but dissolved.

The content of the crystalline form of the cannabidiol in the spray dried powders according to the present invention can be measured using commonly known methods, such as differential scanning calorimetry (DSC).

In the context of the present invention, DSC is used to determine the amount of crystalline cannabidiol in the spray dried powders according to the present invention according to standard methods in the art, i.e. by using crystalline cannabidiol as external standard.

The DSC method is carried out as follows:

The spray dried powder (ca. 8-10 mg) is placed in aluminium pans and hermetically sealed. Empty pans were used as a reference samples. For the investigation of the crystalline cannabidiol bulk material, about 1-2 mg of sample was used (external standard). Each sample was heated from 10° C. at 10 C/min to 90° C. and then cooled down to 10° C. at 10 C/min. Upon heating, for the crystalline cannabidiol the thermal transition peak was determined to be at ca. 68° C., which was attributed to the melting of the cannabidiol crystals. By analysing the DSC thermograms and measuring the peak areas, the degree of crystallinity of a sample can then be determined, as the peak area of the DSC peak corresponds to the amount of heat released during crystallization/heat consumed during melting and is proportional to the degree of crystallinity of a sample. To determine the % of crystallinity, the peak areas of the samples are referenced against the peak area of the crystalline cannabidiol. The amount of crystalline cannabidiol present in the respective sample can then be calculated, which is then used to determine the degree of crystallinity (%), based on known total amount of cannabidiol present in the sample.

The spray dried powder according to the present invention can optionally comprise small amount, such as amounts up to 1 wt.-%, based on the total weight of the spray dried powder, of dyestuffs, flavouring agents and/or sweetening agents.

The spray dried powders according to the present invention are obtainable by a process comprising the consecutive steps of
(i) preparation of a lipid phase by solubilizing the cannabidiol in the Vitamin E, preferably at temperatures of 60 to 100° C., more preferably of 65 to 90° C., most preferably of 70 to 80° C., followed by the addition of tocopherol polyethylene glycol succinate,
(ii) preparation of an aqueous phase by solubilising the gelatine and the hydrolysed starch in the water, followed by
(iii) emulsification of the lipid phase into the aqueous phase, preferably at temperatures of 60 to 100° C., more preferably of 65 to 90° C., most preferably of 70 to 80° C. to form an emulsion followed by
(iv) spray-drying the emulsion.

The spray drying can be performed according to standard methods in the art. It is well understood by a person skilled in the art that the spray dried powders comprise the cannabidiol.

Thus, particularly preferred spray dried powders (A), (A′), (A″), (A-I), (A-I′), (A-I″), (A-II), (A-II′), (A-II″), (A-III), (A-III′) and (A-III″) according to the present invention consist essentially of:
(i-I) 35-42 wt.-%, based on the total weight of the spray dried powder, of cannabidiol,
(ii-I) 5 to 15 wt.-%, based on the total weight of the spray dried powder, of (all-rac)-α-tocopherol,
(iii-I) 3 to 6 wt.-%, based on the total weight of the spray dried powder, of tocopherol polyethylene glycol succinate,
(iv-I) 25 to 36 wt.-%, based on the total weight of the spray dried powder, of pork gelatine having a bloom value selected in the range from 100 to 200,
(v-I) 4 to 10 wt.-%, based on the total weight of the spray dried powder, of hydrolysed starch having a DE value selected in the range of 17-21,
(vi-I) 1 to 3 wt. %, based on the total weight of the spray dried powder, of ascorbic acid, and
(vii-I) 1-3 wt.-%, based on the total weight of the spray dried powder, of water
   and are more preferably further defined by one or more of the following limitations (a) to (e):
(a) wherein the ratio of the at least one cannabinoid to Vitamin E in the spray dried powder is selected in the range from 3:1 to 4:1, and/or
(b) wherein the ratio (w/w) of the gelatine to tocopherol polyethylene glycol succinate in the spray dried powder is selected in the range from 7:1 to 6:1, and/or
(c) wherein the emulsion inner droplet size (D50) in the spray dried powder is less than 150 nm as measured by laser diffraction (Malvern), and/or
(d) wherein the crystallinity of the cannabidiol in the spray dried powder is less than 2.0% (measured by DSC), and/or
(e) wherein at least 90 wt.-%, based on the total weight of the cannabidiol, of the cannabidiol in the spray dried powder is in a non-crystalline (amorphous) form.

Most preferably, the spray dried powders (A), (A′), (A″), (A-I), (A-I′), (A-I″), (A-II), (A-II′), (A-II″), (A-III), (A-III′), and (A-III″) consist essentially of (i-I) to (vii-I) and are further characterized by (a) and (b) and one of (d) or (e), preferably by (a) to (c) and one of (d) or (e), most preferably by all of (a) to (e).

In some embodiments, compositions disclosed herein comprise cannabidiol or salt thereof in a total amount of between about 0.1% and about 70% by weight of the composition, for example about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, or about 70%.

The pharmaceutical compositions according to the present disclosure may be formulated into a variety of dosage forms, such as a tablet (orally dispersible tablet, chewable tablet, film coated tablet, immediate release tablet, extended or sustained release tablet and similar), a capsule, an orally dispersible film, or a gummy. Such dosage forms may each comprise between about 10 mg to about 1500 mg of cannabidiol or pharmaceutically acceptable salt thereof, or about 25 mg to about 1200 mg, or about 50 mg to about 1000 mg, or about 75 mg to about 750 mg, or about 100 mg to about 500 mg, or about 125 mg to about 400 mg, or about 150 mg to about 300 mg.

### D. METHODS OF TREATMENT

In one embodiment, the pharmaceutical composition of the present invention is for use in reducing acute or chronic pain, e.g., for post-operative pain, for reducing pain associated with or treating and/or preventing conditions like arthritis, multiple sclerosis, and neuropathic pain. In a further embodiment, the pharmaceutical composition of the present invention is for use in reducing anxiety, particularly in people with generalized anxiety disorder (GAD), social anxiety disorder (SAD), and post-traumatic stress disorder (PTSD). In a further embodiment, the pharmaceutical composition of the present invention is for use in treating or preventing neurodegenerative diseases like Alzheimer's and Parkinson's. In a further embodiment, the pharmaceutical composition of the present invention is for use in treating autoimmune conditions, such as rheumatoid arthritis and Crohn's disease. In a further embodiment, the pharmaceutical composition of the present invention is for use in improving sleep quality and/or regulating sleep cycles, particularly in individuals with insomnia or those suffering from anxiety-related sleep disturbances. In a further embodiment, the pharmaceutical composition of the present invention is for use in the treatment of diseases of the central nervous system, including multiple sclerosis, amyotrophic lateral sclerosis, Parkinson's disease, Huntington's disease, autism, and schizophrenia; opioid use disorders; Tourette's syndrome; glaucoma; IBD; IBS; as well as cancer and cancer related illnesses, including gliomas, glioblastomas, colorectal cancer, pancreatic cancer, lung cancer, and breast cancer.

The pharmaceutical composition is to be administered to the subject to provide an effective amount of cannabidiol as the active moiety. The effective amount of cannabidiol to be administered to a subject in need thereof is generally selected in the range of about 10 mg to 1500 mg/day, respectively up to about 20 mg/kg/day; for example,
up to 150 mg/day for improving wellness;
up to 300 mg/day for treating anxiety and/or stress;
up to 1000 mg/day for treating pain;
up to 300 mg/day for treating sleeping disorders;
up to 1500 mg/day for treating epilepsy and seizures.

In certain embodiments, the pharmaceutical compositions can be administered to patients having the following conditions:
Seizures Associated with Lennox-Gastaut Syndrome or Dravet Syndrome-starting dosage is 2.5 mg/kg by mouth twice daily (5 mg/kg/day). After one week, the dosage can be increased to a maintenance dosage of 5 mg/kg twice daily (10 mg/kg/day). Based on individual clinical response and tolerability, the dose can be increased up to a maximum recommended maintenance dosage of 10 mg/kg twice daily (20 mg/kg/day).
Seizures Associated with Tuberous Sclerosis Complex-starting dosage is 2.5 mg/kg by mouth twice daily (5 mg/kg/day). Increase the dose weekly by 2.5 mg/kg twice daily (5 mg/kg/day), as tolerated, to a recommended maintenance dosage of 12.5 mg/kg twice daily (25 mg/kg/day).

In a preferred embodiment, the pharmaceutical composition of the present invention is for use in therapy and/or for use in the treatment of a patient in need thereof (e.g., a patient suffering from any of the diseases or conditions mentioned above), wherein the administration of the pharmaceutical composition independent from the patient's state of metabolism. Hence, the patient can be either in the fasted or fed state.

When used to treat pain, inventive compositions can have an opioid-sparing effect in managing patients' pain, i.e., no or a decreased amount of opioid is needed to alleviate the pain. Thus, in certain embodiments, the CBD compositions herein are approximately equally-effective in treating pain as compared to an opioid. Furthermore, the inventive CBD compositions are advantageous for use pre- or post-operatively in which the patients are typically in a fasted state, i.e., the inventive compositions have superior pharmacokinetic properties in fasted patients as compared to other CBD preparations (e.g., Epidyolex).

In another embodiment, the present disclosure is to a method of treating acute or chronic pain by administering to a patient in need thereof a therapeutically effective dose of a pharmaceutical composition comprising:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of a Vitamin E Derivative; and
(iv) one or more excipients or additives in an amount to make the composition total 100 wt.-%;
   wherein the patient's pain is effectively treated without the concomitant administration of an opioid analgesic, and without regard to the proximity of a meal. For instance, the pharmaceutical composition can be administered at a cannabidiol dose of about 5 mg/kg/day to 20 mg/kg/day as the sole pharmaceutical intervention to treat the pain. In another embodiment, such composition is administered in a fasted state and achieves therapeutically effective pharmacokinetic values sufficient to treat the pain, wherein the pain is measured by the Visual Analogue Scale (VAS). VAS is a validated, subjective measure for acute and chronic pain. Scores are recorded by making a handwritten mark on a 10-cm line that represents a continuum between “no pain” and “worst pain.” See Delgado et al, “Validation of Digital Visual Analog Scale Pain Scoring With a Traditional Paper-based Visual Analog Scale in Adults,” J. Am. Acad. Orthop. Surg. Glob. Res. Rev. Vol. 23; 2(3) (March 2018). In one embodiment, the composition is administered to a patient before or after a dental procedure, for example oral surgery, under fasted conditions, and the pain is successfully managed as confirmed by VAS assessment.

In other embodiments, the inventive composition can be co-administered with an opioid resulting in a synergistic effect in treating the pain, i.e., the combined effect of the CBD composition with an opioid is greater than the sum of their individual effects. Accordingly, in one embodiment, the pain is successfully treated (as measured by VAS) with a combination of CBD composition and opioid but at lower individual doses of each. For example, the CBD is dosed at 5 mg/kg/day to 15 mg/kg/day and the opioid is dosed at 20% to 80% of its effective dose if given alone.

The opioids and reported equianalgesic oral dosages (per dosing interval) when used as monotherapy are:

| Active Agent | Oral dose (mg) |
|---|---|
| Codeine | 200 |
| Hydrocodone | 30-45 |
| Hydromorphone | 7.5 |
| Levorphanol | 4 |
| Meperidine | 300 |
| Methadone | 20 |
| Morphine | 30 |
| Oxycodone | 20-30 |
| Oxymorphone | 15 |
| Pentazocine | 180 |

MSD Manual Professional Version (2025); NIH Report (accessed Aug. 14, 2025 at: www.ncbi.nlm.nih.gov/books/NBK65949.1/table/CDR0000062738_557/?report=objectonly).

Sample initial doses for opioid naïve adults when used as monotherapy include:
Oxycodone immediate release: 5 to 10 mg orally every 3 to 4 hours.
Hydromorphone immediate release: 2 to 4 mg orally every 3 to 4 hours.
Hydrocodone immediate release: 5 to 10 mg orally every 4 to 6 hours.
Morphine immediate release: 7.5 to 15 mg orally every 3 to 4 hours.
Tramadol immediate release: 50 to 100 mg orally every 4 to 6 hours.
Tapentadol immediate release: 50 to 100 mg orally every 4 to 6 hours.
Codeine immediate release: 15 to 60 mg orally every 4 to 6 hours.
Wolters Kluwer, Opioids Commonly Used for Acute Pain in the Ambulatory Setting, 2025 (accessed on Aug. 14, 2025 at: www.uptodate.com/contents/image?imageKey=ANEST % 2F142435).

Accordingly, such doses of the opioids may be decreased when combined with the CBD compositions herein such as, for example, by about 20% to 80%. In certain embodiments, the use of such opioids can be avoided altogether or used in combination with the inventive formulations but as lower dosages. For instance, the combination of the CBD composition with an opioid has an additive effect, i.e., the combined effect is equal to the sum of the individual effects.

Another application of the present invention is a method of treating insomnia or other sleep disorder in a patient in need thereof, comprising administering a pharmaceutical composition comprising:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of a Vitamin E Derivative; and
(iv) one or more excipients or additives in an amount to make the composition total 100 wt.-%;
   wherein a therapeutically effective amount of the composition is administered within about 60 minutes after a meal, and wherein compared to an equivalent dose of Epidyolex, the composition has a Tₘₐₓ that is about 2.5%, or about 5%, or about 10%, or about 15%, or about 20%, or about 25%, or about 30%, or about 40%, or about 50%, or about 60% faster than Epidyolex.

In another embodiment, the invention relates to a kit of parts for administering the pharmaceutical composition according to the present invention to a subject in need thereof, comprising: a) the pharmaceutical composition with all the definitions and preferences as provided herein; and b) instructions for dosing the pharmaceutical composition, wherein the instructions specify the administration of the pharmaceutical composition to the subject can occur either in the fed or the fasted state.

In further aspects, the present invention relates to methods of treatment involving the above-described pharmaceutical compositions, spray dried powders, and uses, comprising orally administering to a patient in need thereof a composition of the present invention, wherein the patient is suffering from a disease or condition comprising chronic pain; neuropathic pain; post-operative pain (e.g., dental procedure or oral surgery); anxiety, including generalized anxiety disorder (GAD), social anxiety disorder (SAD), and post-traumatic stress disorder (PTSD); neurodegenerative diseases, including Alzheimer's and Parkinson's; autoimmune conditions, including rheumatoid arthritis and Crohn's disease; sleep disorders, including insomnia and anxiety-related sleep disturbances; diseases of the central nervous system, including multiple sclerosis, amyotrophic lateral sclerosis, Parkinson's disease, Huntington's disease, autism, and schizophrenia; opioid use disorders; Tourette's syndrome; glaucoma; IBD; IBS; and cancer and cancer related illnesses, including gliomas, glioblastomas, colorectal cancer, pancreatic cancer, lung cancer, and/or breast cancer.

### E. PHARMACOKINETICS

The oral pharmaceutical compositions described herein have advantageous pharmacokinetic characteristics. The compositions, namely those comprising:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of a Vitamin E Derivative; and
(iv) one or more excipients or additives in an amount to make the composition total 100 wt.-%.
   will exhibit the following properties after oral administration.

### Fasted Conditions:

Tₘₐₓ—about 2 to 6 hours, or about 3 to 5 hours, or about 3.5 to 4.5 hours, or about 4 hours. Compared to an equivalent dose of Epidyolex, the composition has a Tₘₐₓ that is about 2.5%, or about 5%, or about 10%, or about 15%, or about 20%, or about 25%, or about 30%, or about 40%, or about 50%, or about 60% faster than Epidyolex.
Cₘₐₓ—after a dose of about 400 mg cannabidiol, a Cₘₐₓ of about 90 to 300 ng/ml, or about 110 to 250 ng/ml, or about 150 to 225 ng/ml, or about 200 ng/ml. Compared to an equivalent dose of Epidyolex, the composition has a Cₘₐₓ that is about 2.5%, or about 5%, or about 10%, or about 15%, or about 20%, or about 30%, or about 40%, or about 50%, or about 60% higher than Epidyolex.
AUC—after a dose of about 400 mg cannabidiol, a CBD AUC₀₋₂₄ of about 500 to 900, or about 550 to 850, or about 600 to 800, or about 700 h*ng CBD equivalents/mL, and a 7—OH-CBD AUC₀₋₂₄ of about 400 to 700, or about 450 to 650, or about 500 to 600, or about 550 h*ng CBD equivalents/mL (the AUC₀₋₂₄ of 7-OH-CBD is multiplied by 0.9516 to obtain CBD equivalents). Compared to an equivalent dose of Epidyolex, the composition has an AUC₀₋₂₄ (sum of CBD and 7-OH-CBD) that is about 5%, or about 10%, or about 15%, or about 20%, or about 30%, or about 40%, or about 50%, or about 60%, or about 70%, or about 80% higher than Epidyolex.

### Fed Conditions:

Tₘₐₓ—about 2 to 8 hours, or about 3 to 7 hours, or about 3.5 to 5 hours, or about 4 hours. Compared to an equivalent dose of Epidyolex, the composition has a Tₘₐₓ that is about 2.5%, or about 5%, or about 10%, or about 15%, or about 20%, or about 25%, or about 30%, or about 40%, or about 50%, or about 60% faster than Epidyolex.
Cₘₐₓ—after a dose of about 400 mg cannabidiol, a Cₘₐₓ of about 90 to 150 ng/ml, or about 100 to 145 ng/ml, or about 110 to 140 ng/ml, or about 135 ng/ml. Compared to an equivalent dose of Epidyolex, the composition has a Cₘₐₓ that is about 2.5%, or about 5%, or about 10%, or about 15%, or about 20%, or about 30%, or about 40%, or about 50%, or about 60% higher than Epidyolex.
AUC—after a dose of about 400 mg cannabidiol, a CBD AUC₀₋₂₄ of about 1500 to 2500, or about 1850 to 2250, or about 2000 to 2100, or about 2050 h*ng CBD equivalents/mL, and a 7-OH-CBD AUC₀₋₂₄ of about 400 to 700, or about 450 to 650, or about 500 to 600, or about 550 h*ng CBD equivalents/mL (the AUC₀₋₂₄ of 7-OH-CBD is multiplied by 0.9516 to obtain CBD equivalents). Compared to an equivalent dose of Epidyolex, the composition has an AUC₀₋₂₄ (sum of CBD and 7-OH-CBD) that is about 1%, or about 5%, or about 10%, or about 15%, or about 20%, or about 30%, or about 40% higher than Epidyolex.

In one embodiment, it has been surprisingly found that the oral pharmaceutical composition comprising cannabidiol, Vitamin E and TPGS results in a 15% higher plasma AUC₀₋₂₄ of CBD and a 49% higher plasma AUC₀₋₂₄ of 7-OH CBD (p<0.001), when compared to the existing liquid formulation Epidyolex (i.e. an alcoholic sesame oil CBD composition (liquid composition) which consists essentially of 100 mg (~10.9 wt.-%) CBD, 79 mg (~8.6 wt.-%) EtOH, 736 mg (~80.4 wt.-%) sesame oil and 0.5 mg (~0.05 wt.-%) of sucralose per mL solution), when administered at equal CBD dosage level and when administered in a fasted state.

Therefore, in a preferred embodiment, the pharmaceutical composition of the present invention is for use in therapy and/or for use in the treatment of a patient in need thereof (e.g., a patient suffering from any of the diseases or conditions mentioned above), wherein the patient is in a fasted state.

In another preferred embodiment, the invention relates to a kit of parts for administering the pharmaceutical composition according to the present invention to a subject in a fasted state, comprising a) the pharmaceutical composition with all the definitions and preferences as provided herein; and b) instructions for dosing the pharmaceutical composition, wherein the instructions specify the administration of the pharmaceutical composition to the subject in a fasted state.

In another preferred embodiment, the pharmaceutical composition according to the present invention provides an AUC₀₋₂₄ of CBD and/or of 7-OH CBD which is at least 10% higher (for example, at least 15% higher) compared to that of an alcoholic oil solution of CBD such as preferably Epidyolex when administered in a fasted state. In a further embodiment, the pharmaceutical composition according to the present invention has an AUC₀₋₂₄ of 7-OH CBD in a fasted state that is at least 20% higher, most preferably at least 30% or 40% higher, as compared to an alcoholic oil solution of CBD such as preferably Epidyolex.

In another aspect, the invention is directed to the use of an oral pharmaceutical composition according to the present invention to enhance the bioavailability of CBD (including its biologically active metabolite 7-OH CBD) in a fasted state, preferably by at least 10%, more preferably by at least 15% or at least 20%, most preferably by at least 30% when compared to an alcoholic oil solution of CBD such as preferably Epidyolex. It is well understood that said use encompasses the incorporation of CBD in a spray dried powder with all the definitions and preferences as provided herein.

Surprisingly, it has also been found that the oral pharmaceutical composition according to the present invention provides a higher Cₘₐₓ and a significantly earlier Tₘₐₓ of CBD in plasma, when compared to the existing liquid formulation Epidyolex (i.e. an alcoholic sesame oil CBD composition (liquid composition), which consists essentially of 100 mg (~10.9 wt.-%) CBD, 79 mg (~8.6 wt.-%) EtOH, 736 mg (~80.4 wt.-%) sesame oil and 0.5 mg (~0.05 wt.-%) of sucralose per mL solution), when administered at equal CBD dosage level and when administered in a fed state.

Therefore, in a preferred embodiment, the pharmaceutical composition of the present invention is for use in therapy and/or for use in the treatment of a patient in need thereof (e.g., a patient suffering from any of the diseases or conditions mentioned above), wherein the patient is in a fed state.

In another embodiment, the invention relates to a kit of parts for administering the pharmaceutical composition according to the present invention to a subject in a fed state, comprising: a) the pharmaceutical composition with all the definitions and preferences as provided herein; and b) instructions for dosing the pharmaceutical composition, wherein the instructions specify the administration of the pharmaceutical composition to the subject in a fed state.

The present inventors found that the oral pharmaceutical composition according to the present invention has an earlier Tₘₐₓ of CBD compared to an alcoholic oil solution of CBD such as preferably Epidyolex when administered in a fed state.

In pharmacokinetics, Tₘₐₓ refers to the time it takes for a drug to reach its maximum concentration (Cₘₐₓ) in the bloodstream after administration. The Cₘₐₓ is the highest concentration of the drug observed during the absorption phase. An earlier Tₘₐₓ is beneficial when a rapid therapeutic effect is needed. If Tₘₐₓ occurs early, the drug reaches its maximum concentration faster, resulting in quicker onset of action. This is particularly important for drugs designed to have fast-acting effects to provide relief in acute situations. Achieving Tₘₐₓ earlier also allows clinicians to more quickly assess if Cₘₐₓ has reached the desired therapeutic level. This helps in adjusting the dose promptly to avoid underdosing or overdosing and unwanted side effects.

Thus, in a preferred embodiment, the oral pharmaceutical composition according to the present invention has a Tₘₐₓ of CBD in the range of 2 to 6 hours, preferably in the range of 4 to 6 hours, most preferably in the range of 5 to 6 hours, when administered in a fed state. In another embodiment, the oral pharmaceutical composition according to the present invention has an earlier Tₘₐₓ for CBD than an alcoholic oil solution of CBD such as preferably Epidyolex when administered in a fed state.

In another aspect, the invention is directed to the use of an oral pharmaceutical composition according to the present invention to improve the Tₘₐₓ of CBD in a fed state, preferably by at least 1 hour, more preferably by at least 2 hours, most preferably by at least 3 hours when compared to an alcoholic oil solution of CBD such as preferably Epidyolex. It is well understood that said use encompasses the incorporation of CBD in a spray dried powder with all the definitions and preferences as provided herein.

It has also been found that the oral pharmaceutical composition according to the present invention provides a higher Cₘₐₓ of CBD compared to an alcoholic oil solution of CBD such as preferably Epidyolex when administered in a fed state. Thus, in one embodiment, the oral pharmaceutical composition according to the present invention has a higher Cₘₐₓ for CBD than an alcoholic oil solution of CBD such as preferably Epidyolex.

Further, it was found that an oral pharmaceutical composition comprising the spray dried CBD powder according to the present invention has improved pharmacokinetic properties with respect to variability when compared to an alcoholic oil solution of CBD such as preferably Epidyolex. As shown in the study described herein, the coefficient of variation (CV) for AUC₀₋₂₄ and Cₘₐₓ was substantially lower for the inventive composition in the fed state.

Therefore, in a preferred embodiment, the oral pharmaceutical composition according to the present invention has a coefficient of variation (CV) of CBD in a fed state that is lower compared to that of an alcoholic oil solution of CBD such as preferably Epidyolex. In another preferred embodiment, the oral pharmaceutical composition according to the present invention has a CV of CBD that is below 45%, when administered in a fed state.

Further, it was found that an oral pharmaceutical composition comprising the spray dried CBD powder according to the present invention has improved pharmacokinetic properties with respect to variability when compared to the existing liquid formulation Epidyolex (i.e. an alcoholic sesame oil CBD composition (liquid composition), which consists essentially of 100 mg (~10.9 wt.-%) CBD, 79 mg (~8.6 wt.-%) EtOH, 736 mg (~80.4 wt.-%) sesame oil and 0.5 mg (~0.05 wt.-%) of sucralose per mL solution) when administered at equal CBD dosage level. As shown in the study described herein, the coefficient of variation (CV) for AUC₀₋₂₄ and Cₘₐₓ was substantially lower for the inventive composition, both in fasted and fed state.

When a new drug formulation shows reduced variability, as indicated by a lower coefficient of variation (CV) compared to a known drug formulation, several advantages arise in terms of consistent drug action, patient safety, dosing precision, and overall treatment reliability. This can lead to improved patient outcomes, enhanced treatment adherence, and broader acceptance in both clinical and regulatory environments.

Therefore, in a preferred embodiment, the oral pharmaceutical composition according to the present invention has a coefficient of variation (CV) of CBD that is lower compared to that of an alcoholic oil solution of CBD such as preferably Epidyolex. In another preferred embodiment, the oral pharmaceutical composition according to the present invention has a CV of CBD that is below 83%, when administered in a fasted state (preferably below 80%, more preferably below 75%, even more preferably below 70%); and/or the oral pharmaceutical composition according to the present invention has a CV of CBD that is below 45%, when administered in a fed state.

In another aspect, the invention is directed to the use of an oral pharmaceutical composition according to the present invention to reduce the coefficient of variation (CV) of CBD, irrespective of the metabolic state of the patient to which the pharmaceutical composition is to be administered.

Such use may comprise the step of orally administering 400 mg of cannabidiol in the form of one or more capsules, wherein the coefficient of variation (CV) of CBD is below 83%, (preferably below 75%, more preferably below 70%) when the patient is in the fasted state; and/or the CV of CBD is below 45% when the patient is in the fed state. The method of treatment or use may include the step of orally administering without specifying whether the patient is in the fed or fasted state, or wherein the step of orally administering is performed without regard to proximity to a meal.

Alternatively, the step of orally administering may be performed with specifying consistent administration in either the fed or fasted state, and specifying that a missed dose may be taken without regard to proximity to a meal, or wherein the step of orally administering is performed with specifying consistent administration with or without food and specifying that a missed dose may be taken without regard to proximity to food.

The present invention encompasses formulations that are bioequivalent to the pharmaceutical compositions described herein, such as the CBD capsules detailed in Table 1 below. Accordingly, in one embodiment, the present disclosure is directed to a pharmaceutical composition that is bioequivalent to a formulation comprising:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of a Vitamin E Derivative; and
(iv) one or more excipients or additives in an amount to make the composition total 100 wt.-%;
   wherein bioequivalence is established by (a) a 90% Confidence Interval (CI) for AUC which is between 0.80 and 1.25; and (b) a 90% CI for Cₘₐₓ which is between 0.80 and 1.25. In another embodiment, the bioequivalence is tested under fasted conditions, and the formulation is the CBD capsules in Table 1 below.

### F. EXAMPLES

The present invention is further illustrated by the following examples. The examples are intended to illustrate, not to limit, the disclosed embodiments.

### Example 1

### 1.1 Preparation of CBD Spray Dried Powder (CBD-SDP)

A spray dried composition comprising cannabidiol (CBD) was prepared.
(i) For the preparation of the lipid phase, a total of 66.68 g of CBD (Brains Bioceutical, United Kingdom) was dispersed in 28.58 g of (all-rac)-α-tocopherol (dsm-firmenich, Switzerland) at 75° C. under stirring until complete dissolution. Subsequently, 9.53 g of tocopherol polyethylene glycol succinate (Vitamin E TPGS (Merck, Germany)) was added to the warm (75° C.) CBD/(all-rac)-α-tocopherol mixture and homogenized.

The lipid phase was kept warm until completing dispersion into the aqueous phase.
(ii) For the preparation of the aqueous phase, a total of 63.8 g pork gelatine, bloom 140 (Gelita, Germany), was hydrated and dissolved in 152 g warm water (40° C.). Subsequently, 13.33 g maltodextrin Glucidex 19IT (Roquette, France) and 4.75 g of L-ascorbic acid (dsm-firmenich, Switzerland) was added and the solution was heated to process temperature of 75° C.
(iii) The warm aqueous phase was stirred, and the warm lipid phase was slowly added and homogenized at an elevated temperature (ca. 75° C.) to form a liquid formulation. Afterwards, the emulsion was spray dried at around 180° C. inlet temperature, 80° C. outlet temperature resulting in a white free flowing powder.

The CBD spray dried powder (CBD-SDP) exhibited a CBDHQ content of <0.1% (w/w) as determined by HPLC-DAD and a PSD D10/D50/D90 (μm): 13.0/36.7/127.0 (Malvern).

The CBD in the CBD spray dried powder (CBD-SDP) was in a substantially non-crystalline form (crystallinity as determined by DSC was <2%).

### 1.2 Preparation of Capsules Comprising the CBD Spray Dried Powder

Hard gelatin opaque white capsules size 0 were used for the administration of the CBD spray dried powder (CBD-SDP) of Example 1.1. The CBD-SDP was admixed with mannitol (Merck, Germany) and silicon dioxide (Merck, Germany) in the amounts as outlined in Table 1 to form the capsule mixture. The capsule mixture was then encapsulated in the gelatin capsules using an automatic filling machine at the temperature <25° C. and the relative humidity <30%. The target capsule fill weight was 221 mg.

The resulting CBD capsules were composed as shown in Table 1 below.

TABLE 1
Composition of capsules comprising the
CBD spray dried powder of Example 1.1

| | Formulation | |
|---|---|---|
| | Capsule Fill | |
| | Quantity | |
| Component and Quality Standard | per unit | % |
| (and Grade, if applicable) | (mg) | (w/w) |
| Cannabidiol (CBD) (DAC) | 66.68 | 30.17 |
| All-rac-α-tocopherol (Ph. Eur./USP-NF) | 28.58 | 12.93 |
| Tocopherol polyethylene glycol succinate | 9.53 | 4.31 |
| (USP-NF) | | |
| Porcine gelatine 140 bloom (Ph. Eur. Chapter | 63.8 | 28.87 |
| 5.2.8/USP-NF) | | |
| Maltodextrin Glucidex 19IT (Ph. Eur./USP-NF) | 13.33 | 6.03 |
| L-ascorbic acid (USP-NF, FCC, Ph. Eur.) | 4.75 | 2.15 |
| Silicon dioxide CAS 7631-86-9 | 2.21 | 1 |
| Mannitol | 28.31 | 12.81 |
| Water | 3.80 | 1.72 |
| Total | 221 mg | 100 |

### Example 2—Comparative Pharmacokinetic Study in Humans

A pharmacokinetic study was conducted to evaluate the CBD capsules of example 1.2 (in the following referred to as test product (TP)) under both fed and fasted conditions, and comparing its performance to the liquid CBD reference product (RP) Epidyolex. The study followed a randomized, open-label, 4-way cross-over design and involved 32 healthy subjects aged 19 to 55 years, with an equal number of men and women. The study was conducted at Apex Trials, Nutrasource, 120 Research Lane, Suite 203, Guelph, Ontario N1G 0B4, Canada, a contract research organization.

400 mg CBD were administered to each participant. The test product (TP) were the CBD capsules of Example 1.2, while the reference product (RP) was a liquid alcoholic oil solution of CBD which is commercially available as Epidyolex® (GW Pharma, The Netherlands). Participants received each of the following four treatments: fed condition with TP, fed condition with RP, fasted condition with TP, and fasted condition with RP, with washout periods of at least 14 days between each treatment.

Participants fasted overnight for a minimum of 10 hours prior to the administration of either TP or RP. TP or RP was then administered i) in fed state, after a standardized FDA high fat/calorie breakfast of 800 to 1′000 kcal which was consumed within a timeframe of 30 minutes, or ii) in fasted state, after the 10 hours of overnight fastening plus another 30 minutes of fasting.

Blood samples were taken at multiple time points, including 0, 0.25, 0.5, 0.75, 1, 1.5, 2, 3, 4, 5, 6, 8, 12, and 24 hours post-dose. The study determined CBD, 7-OH-CBD, and 7-COOH)-CBD in plasma. Key pharmacokinetic parameters were used to assess the bioavailability differences. The parameters measured were area under the curve from 0 to 24 hours (AUC₀₋₂₄), maximum concentration reached (Cₘₐₓ), and time to reach Cₘₐₓ (Tₘₐₓ). Additionally, the sums of AUC₀₋₂₄ for CBD and 7-OH-CBD in [h*ng CBD equivalents/mL] for direct comparison of the different molecules (with different molecular weights) were calculated and compared for the two different CBD products (TP and RP) after a single administration.

The study provided valuable insights into the pharmacokinetics of the pharmaceutical composition of the present invention compared to the liquid reference product Epidyolex, evaluating its performance in different conditions.

As the test product (TP) for the study, hard gelatin capsules size 0 comprising the CBD spray dried powder were used. The capsules were prepared as described in Example 1.2 above, and each contained 66.67 mg of CBD. Six capsules were administered per participant which corresponds to 400 mg of CBD per administration.

The reference product (RP) used for the study was Epidyolex®, a liquid CBD formulation containing 100 ml/mg of CBD dissolved in sesame oil and anhydrous ethanol. 4 ml were administered per participant which corresponds to 400 mg of CBD per administration. The product was purchased at a Swiss pharmacy (TopPharm Apotheke, 4153 Reinach, Switzerland) for clinical trial research purpose in accordance with the BAG (Bundesamt für Gesundheit) regulations. Further information regarding Epidyolex can be taken from Table 2.

TABLE 2
Information on Epidyolex ®

| | |
|---|---|
| Proprietary (Brand) Name of Drug | Epidyolex ® |
| Product | |
| Non-proprietary or Common Name | Cannabidiol |
| of Drug Substance (Medicinal | |
| Ingredient) | |
| Company Name and Address of | GW Pharma (International) B.V., |
| Manufacturer | Databankweg 26, 3821AL |
| | Amersfoort, The Netherlands |
| | e-mail: |
| | medinfo-int@jazzpharma.com |
| Dosage Form(s) | Oral solution |
| Strength(s) | 100 mg/ml |
| Lot Number | LOT: 115596 |
| Expiration Date | EXP: February 2025 |
| Country the Reference Drug Product | Switzerland |
| is Marketed in for the Lot to be | |
| Used in this Clinical Trial | |

### Study Results

400 mg CBD as TP or RP were administered to the participants in fasted and fed state.

For fasted state assessment, 400 mg CBD as TP or RP were administered to each participant after an overnight fast of at least 10 plus 0.5 hours. CBD concentration in plasma was then measured for 24 hours. For each participant, their individual Cₘₐₓ, Tₘₐₓ, and AUC₀₋₂₄ for the TP and RP were derived from the plasma curve.

In line with FDA statistical guidance for pharmacokinetics studies, the Cₘₐₓ and AUC₀₋₂₄ values were log-transformed and then statistically analysed to estimate the overall ratio between TP and RP. In the model, treatment sequence, treatment, and period were treated as fixed effects and subject nested within sequence as a random effect. Body weight was used as covariate. Geometric mean (GM), Geometric mean ratio (GMR), as well as p-values and 90% confidence intervals (CI) of GMR were calculated by exponentiating Least square means (LSM) and the difference in LSM between TP and RP.

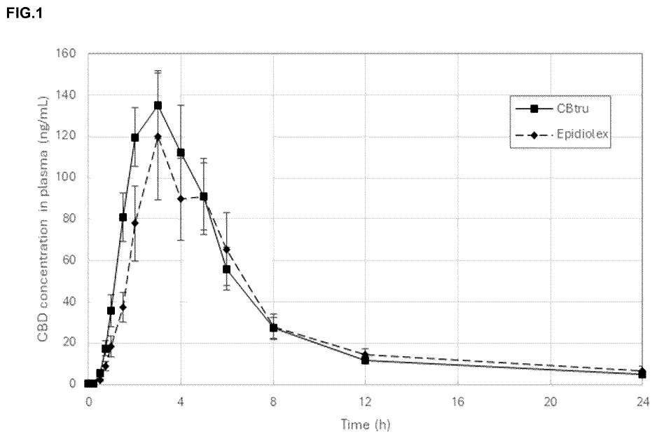

In fasted state, the AUC₀₋₂₄ of cannabidiol was 1.15 times higher with the test product (TP) than with the reference product RP (geometric mean ratio), and the Cₘₐₓ was 1.35 times higher (geometric mean ratio). Hence, the TP resulted in 15% higher bioavailability over 24 hours and reached a 35% higher peak. That is, with the TP, more of the ingested 400 mg of CBD was found in the plasma over time than with the RP (see FIG. 1).

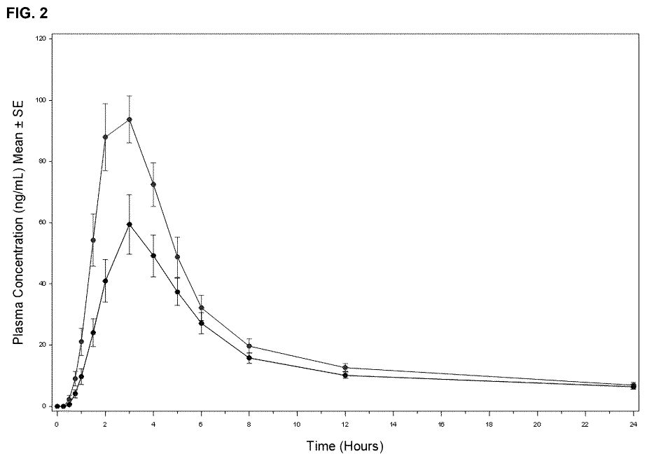

The geometric mean ratio of AUC₀₋₂₄ (TP to RP) for 7-OH-CBD (a biologically active metabolite of CBD) was 1.49. Hence, the bioavailability of the metabolite was 49% higher with the TP compared to the RP (p<0.001). The GMR of Cₘₐₓ (TP to RP) was 1.85, i.e. the TP was 85% higher (p<0.001). Tₘₐₓ also occurred earlier in the TP (p<0.048). These results indicate a significantly earlier and higher presence of 7-OH-CBD in the blood with the TP compared to the RP (see FIG. 2 and Table 3).

TABLE 3
Plasma Levels of CBD and its metabolite 7-OH-CBD, reflecting
the total amount of biological actives in the blood over a period
of 24 h. The AUC₀₋₂₄ of 7-OH-CBD was multiplied by 0.9516 to
obtain CBD equivalents. The results are illustrating the surprising
effect of the pharmaceutical composition according to the present
invention in the fasted condition, i.e. an improvement by more
than 19% compared to Epidyolex, while in the fed state no relevant
differences were observed.

| CBD | | Fasted | Fed |
|---|---|---|---|
| Product | Active | AUC0-24 h [h*ng CBD equivalents/mL] | |
| TP | CBD | 771.2 | 2033.0 |
| | 7-OH CBD | 533.9 | 577.1 |
| | Sum | 1305.1 | 2610.1 |
| RP | CBD | 717.4 | 2132.7 |
| | 7-OH CBD | 378.0 | 549.6 |
| | Sum | 1095.4 | 2682.3 |

For fed state assessment, participants arrived in the morning after an overnight fast of at least 10 hours, then had to eat a high-fat, high-calorie standardized FDA breakfast (800-1′000 kcal) within 30 minutes, followed by an intake of a 400 mg CBD dose of test product (TP) or reference product (RP, Epidyolex). CBD concentration in plasma was then measured for 24 hours. For each participant their individual Cₘₐₓ, Tₘₐₓ and AUC₀₋₂₄ for the TP and RP were derived from the plasma curve over 24 h.

In line with FDA statistical guidance for pharmacokinetics studies, the Cₘₐₓ and AUC₀₋₂₄ values were log-transformed and then statistically analyzed to estimate the overall ratio between the TP and RP. In the model, treatment sequence, treatment and period were treated as fixed effects and subject nested within sequence as a random effect. Body weight was used as covariate. Geometric mean (GM) and Geometric mean ratios (GMR were calculated by exponentiating least square means (LSM) and the difference in LSM between the TP and RP.

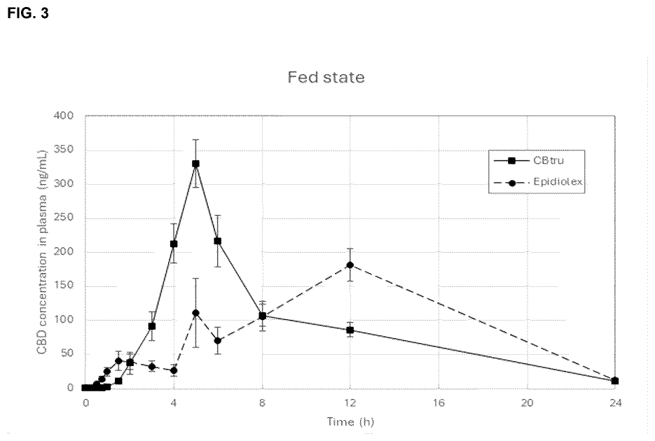

In the fed state, the modelled geometric mean of Cₘₐₓ was 319 ng/ml in the TP, and 254 ng/ml in RP, resulting in a geometric mean ratio of 1.25. Hence, the Cₘₐₓ of the TP was 25% higher. Tₘₐₓ occurred significantly earlier in TP than RP (median 5 h vs 8 h, respectively, p<0.001) (see FIG. 3).

The coefficient of variation (CV) was substantially lower for AUC₀₋₂₄ and Cₘₐₓ, both in fasted and fed state for the test product (TP). The TP thus achieved a more consistent and more reliable plasma response than the reference product (RP; Epidyolex). As regards Tₘₐₓ, the range between minimum and maximum was narrower, 1.5 h to 6.0 h for the TP compared to 2.0 h to 23.5 h for Epidyolex. In the fed state, the interquartile range (from 25th to 75th percentile) was only 5 h to 6 h for the TP, whereas for Epidyolex it was 8 h to 12 h. The results are summarized in Table 4.

TABLE 4
Descriptive statistics of test product (TP) versus reference product
(Epidyolex). Arithmetic mean with standard deviation (SD); coefficient
of variation in % (CV, standard deviation divided by the arithmetic
mean); geometric mean (arithmetic mean on log-transformed data,
then back-transformed); and geometric CV in % (geometric standard
deviation divided by geometric mean); P25 (25^{th} percentile, 1^{st}
quartile, i.e. 25% of all values in the data are below or equal); and
P75 (75^{th} percentile, i.e. 75% of all data are below or equal).

| | TP Fasted | RP Fasted | TP Fed | RP Fed |
|---|---|---|---|---|
| | (N = 30) | (N = 30) | (N = 30) | (N = 32) |
| | CBD AUC₀₋₂₄ (h*ng/mL) | | | |
| Arithmetic Mean | 771 (517) | 717 (594) | 2033 (857) | 2133 (955) |
| (SD) | | | | |
| CV (%) | 67% | 83% | 42% | 45% |
| Geometric mean | 633 | 549 | 1909 | 1911 |
| Geometric CV (%) | 70% | 81% | 35% | 54% |
| | CBD Cₘₐₓ (ng/mL) | | | |
| Arithmetic Mean | 190 (129) | 178 (177) | 377 (223) | 316 (258) |
| (SD) | | | | |
| CV (%) | 68% | 100% | 59% | 82% |
| Geometric mean | 155 | 115 | 324 | 256 |
| Geometric CV (%) | 74% | 117% | 61% | 73% |
| | CBD Tₘₐₓ (h) | | | |
| Arithmetic Mean | 3.1 (1.2) | 4.5 (3.8) | 6.0 (2.6) | 9.4 (4.1) |
| (SD) | | | | |
| Median | 3.0 | 3.5 | 5.0 | 8.0 |
| P25, P75 | 2.0, 4.0 | 3.0, 5.0 | 5.0, 6.0 | 8.0, 12.0 |
| Min, Max | 1.5, 6.0 | 2.0, 23.5 | 3.0, 12.1 | 1.5, 23.6 |

### Example 3—a Pharmacokinetic Study to Compare CBD-NE to Epidyolex in Healthy Adult Volunteers Under Both Fed and Fasted Conditions

A pharmacokinetic study will be conducted to evaluate the CBD capsules of Example 1.2 (CBD-NE) under both fed and fasted conditions in healthy adults and comparing its performance to the liquid CBD reference product Epidyolex (or “TP”). Each participant will receive a dose of each product under both fed and fasted conditions in a crossover design.

Two study products, CBD-NE and Epidyolex, will be administered under both fed and fasted conditions. Participants will be randomized in a 1:1:1:1 ratio to one of 4 treatment sequences. There will be a total of 4 treatment periods consisting of 2 consecutive days, and one washout period. Each dose will be followed by a minimum 14-day, maximum of 28-day washout period, with the last dose followed by a follow-up phone call which will occur within the timeframe of the longest washout period over the study. For the fed state, participants will consume a high-fat, high-calorie breakfast within the 30 minutes prior to dosing.

Pharmacokinetic blood sampling will occur pre-dose and at 0.25 h, 0.5 h, 0.75 h, 1.0 h, 1.5 h, 2.0 h, 3.0 h, 4.0 h, 5.0 h, 6.0 h, 8.0 h, 12.0 h and 24.0 h post-dose.

Blood samples collected will be used to assess the PK profiles of CBD-NE and Epidyolex. PK parameters measured will include maximum concentration in plasma (Cₘₐₓ), time to reach maximum concentration (Tₘₐₓ), elimination half-life (T_{1/2}), area under the plasma concentration-time curve over 24 hours (AUC₀₋₂₄), area under the plasma concentration-time curve from zero to infinity (AUC_{inf}) and AUC₀₋₂₄/AUC_{inf} for CBD, 7-OH-CBD and 7-COOH)-CBD. Safety endpoints will be assessed throughout the study and will include reports of adverse events, 12-lead ECG, vital signs, safety laboratory assessments, and abbreviated physical exam.

The 32 male and female healthy subjects ages 19-55 will be naïve to oral or inhaled cannabis or hemp, or light user of oral or inhaled cannabis or hemp products (not more than 2 times per month on average) who have used cannabis for recreational (non-therapeutic) purposes without severe side effects. The subjects will be able to consume the entire high-fat, high-calorie breakfast provided within 30 minutes at the site during Visits 2 and 4. They will further agree to fast overnight, i.e., no food or liquids (except for water, permitted up to 1 h prior to dosing) for a minimum 10 h prior to starting the high-fat, high-calorie breakfast on Visits 2 and 4 or prior to dosing on Visits 6 and 8.

### Arms and Interventions

| Participant Group/Arm | Intervention/Treatment |
|---|---|
| Experimental: CBD-NE Under | Drug: Cannabidiol (CBD) Powder |
| Fed Condition | 66.68 mg CBD per capsule for a |
| A single dose of CBD-NE (6 | total of 400 mg CBD per serving |
| capsules) following | Other Names: |
| consumption of a high-fat, | CBD-NE |
| high-calorie breakfast | |
| Experimental: TP Under | Drug: Cannabidiol (CBD) Powder |
| Fasted Condition | 66.68 mg CBD per capsule for a |
| A single dose of CBD-NE (6 | total of 400 mg CBD per serving |
| capsules) after a minimum 10- | Other Names: |
| hour fast | CBD-NE |
| Active Comparator: Epidyolex | Drug: Cannabidiol 100 MG/ML |
| Under Fed Condition | [Epidiolex] |
| A single dose of Epidyolex (4 | 100 mg CBD/mL for a total of |
| mL oil) following consumption | 400 mg CBD per serving |
| of a high-fat, high-calorie | Other Names: |
| breakfast | Epidyolex |
| Active Comparator: Epidyolex | Drug: Cannabidiol 100 MG/ML |
| Under Fasted Condition | [Epidiolex] |
| A single dose of Epidyolex (4 | 100 mg CBD/mL for a total of |
| mL oil) after a minimum 10- | 400 mg CBD per serving |
| hour fast | Other Names: |
| | Epidyolex |

### Primary Outcome Measures

The primary outcome measures for this study are:
1. Pharmacokinetic (PK) Properties of CBD-NE compared to Epidyolex under Fed Conditions: Area under the plasma concentration-time curve over 24 hours (AUC₀₋₂₄) for CBD.
2. PK Properties of CBD-NE compared to Epidyolex under Fasted Conditions: AUC₀₋₂₄ for CBD.

### Secondary Outcome Measures

The secondary outcome measures for this study are:
1. PK Properties of CBD-NE compared to Epidiolex under Fed Conditions: AUC0-24 for 7-hydroxycannabidiol (7-OH-CBD) and cannabidiol-7-oic acid (7-COOH)-CBD).
2. PK Properties of CBD-NE compared to Epidyolex under Fasted Conditions: AUC0-24 for 7-OH-CBD and 7-COOH)-CBD.
3. PK Properties of CBD-NE compared to Epidyolex under Fed Conditions: Maximum concentration in plasma (Cmax) for CBD, 7-OH-CBD and 7-COOH)-CBD.
4. PK Properties of CBD-NE compared to Epidyolex under Fasted Conditions: Cmax for CBD, 7-OH-CBD and 7-COOH)-CBD.
5. PK Properties of CBD-NE compared to Epidyolex under Fed Conditions: Time to reach Cmax (Tmax) for CBD, 7-OH-CBD and 7-COOH)-CBD.
6. PK Properties of CBD-NE compared to Epidyolex under Fasted Conditions: Tmax for CBD, 7-OH-CBD and 7-COOH)-CBD.
7. PK Properties of CBD-NE compared to Epidyolex under Fed Conditions: Elimination half-life (T1/2) for CBD, 7-OH-CBD and 7-COOH)-CBD.
8. PK Properties of CBD-NE compared to Epidyolex under Fasted Conditions: T1/2 for CBD, 7-OH-CBD and 7-COOH)-CBD.
9. PK Properties of CBD-NE compared to Epidyolex under Fed Conditions: Area under the plasma concentration-time curve from zero to infinity (AUCinf) for CBD, 7-OH-CBD and 7-COOH)-CBD.
10. PK Properties of CBD-NE compared to Epidyolex under Fasted Conditions: AUC_{inf} for CBD, 7-OH-CBD and 7-COOH)-CBD.
11. PK Properties of CBD-NE compared to Epidyolex under Fed Conditions: Ratio of AUC0-24 to AUC_{inf} for CBD, 7-OH-CBD and 7-COOH)-CBD.
12. PK Properties of CBD-NE compared to Epidyolex under Fasted Conditions: Ratio of AUC0-24 to AUC_{inf} for CBD, 7-OH-CBD and 7-COOH)-CBD.
13. Total Drug Exposure of CBD-NE compared to Epidyolex under Fed Conditions: Sum of AUC0-24 of CBD, 7-OH-CBD and 7-COOH)-CBD.
14. Total Drug Exposure of CBD-NE compared to Epidyolex under Fasted Conditions: Sum of AUC0-24 of CBD, 7-OH-CBD and 7-COOH)-CBD.
15. Total Drug Exposure of CBD-NE compared to Epidyolex under Fed Conditions: Sum of AUCinf of CBD, 7-OH-CBD and 7-COOH)-CBD.
16. Total Drug Exposure of CBD-NE compared to Epidyolex under Fasted Conditions: Sum of AUC_{inf} of CBD, 7-OH-CBD and 7-COOH)-CBD.

### Other Outcome Measures

The other outcome measures for this study are:
1. Comparison of PK Properties of CBD-NE under Fed and Fasted Conditions: AUC0-24 for CBD, 7-OH-CBD and 7-COOH)-CBD.
2. Comparison of PK Properties of Epidyolex under Fed and Fasted Conditions: AUC0-24 for CBD, 7-OH-CBD and 7-COOH)-CBD.
3. Comparison of PK Properties of CBD-NE under Fed and Fasted Conditions: Cmax for CBD, 7-OH-CBD and 7-COOH)-CBD.
4. Comparison of PK Properties of Epidyolex under Fed and Fasted Conditions: Cmax for CBD, 7-OH-CBD and 7-COOH)-CBD.
5. Comparison of PK Properties of CBD-NE under Fed and Fasted Conditions: Tmax for CBD, 7-OH-CBD and 7-COOH)-CBD.
6. Comparison of PK Properties of Epidyolex under Fed and Fasted Conditions: Tmax for CBD, 7-OH-CBD and 7-COOH)-CBD.
7. Comparison of PK Properties of CBD-NE under Fed and Fasted Conditions: T1/2 for CBD, 7-OH-CBD and 7-COOH)-CBD.
8. Comparison of PK Properties of Epidyolex under Fed and Fasted Conditions: T1/2 for CBD, 7-OH-CBD and 7-COOH)-CBD.
9. Comparison of PK Properties of CBD-NE under Fed and Fasted Conditions: AUCinf of CBD, 7-OH-CBD and 7-COOH)-CBD.
10. Comparison of PK Properties of Epidyolex under Fed and Fasted Conditions: AUCinf for CBD, 7-OH-CBD and 7-COOH)-CBD.
11. Comparison of PK Properties of CBD-NE under Fed and Fasted Conditions: Ratio of AUC0-24 to AUC_{inf} for CBD, 7-OH-CBD and 7-COOH)-CBD.
12. Comparison of PK Properties of Epidyolex under Fed and Fasted Conditions: Ratio of AUC0-24 to AUC_{inf} for CBD, 7-OH-CBD and 7-COOH)-CBD.
13. Comparison of Total Drug Exposure of CBD-NE under Fed and Fasted Conditions: Sum of AUC0-24 of CBD, 7-OH-CBD and 7-COOH)-CBD.
14. Comparison of Total Drug Exposure of Epidyolex under Fed and Fasted Conditions: Sum of AUC0-24 of CBD, 7-OH-CBD and 7-COOH)-CBD.
15. Comparison of Total Drug Exposure of CBD-NE under Fed and Fasted Conditions: Sum of AUC_{inf} of CBD, 7-OH-CBD and 7-COOH)-CBD.
16. Comparison of Total Drug Exposure of Epidyolex under Fed and Fasted Conditions: Sum of AUC_{inf} of CBD, 7-OH-CBD and 7-COOH)-CBD.

Applicant expects that the results of the study will demonstrate that CBD-NE has one or more superior PK properties compared to Epidyolex listed below.

### Fasted Conditions:

Tₘₐₓ—For each of CBD and 7-OH-CBD, about 2 to 6 hours, or about 3 to 5 hours, or about 3.5 to 4.5 hours, or about 4 hours. Compared to an equivalent dose of Epidyolex, the composition has a Tmax that is about 2.5%, or about 5%, or about 10%, or about 15%, or about 20%, or about 25%, or about 30%, or about 40%, or about 50%, or about 60% faster than Epidyolex.
Cₘₐₓ—For each of CBD and 7-OH-CBD, after a dose of about 400 mg cannabidiol, a Cₘₐₓ of about 90 to 300 ng/ml, or about 110 to 250 ng/mL, or about 150 to 225 ng/ml, or about 200 ng/ml. Compared to an equivalent dose of Epidyolex, the composition has a Cₘₐₓ that is about 2.5%, or about 5%, or about 10%, or about 15%, or about 20%, or about 30%, or about 40%, or about 50%, or about 60% higher than Epidyolex.
AUC—after a dose of about 400 mg cannabidiol, a CBD AUC₀₋₂₄ of about 500 to 900, or about 550 to 850, or about 600 to 800, or about 700 h*ng CBD equivalents/mL, and a 7-OH-CBD AUC₀₋₂₄ of about 400 to 700, or about 450 to 650, or about 500 to 600, or about 550 h*ng CBD equivalents/mL (the AUC₀₋₂₄ of 7-OH-CBD is multiplied by 0.9516 to obtain CBD equivalents). Compared to an equivalent dose of Epidyolex, the composition has an AUC₀₋₂₄ (sum of CBD and 7-OH-CBD) that is about 5%, or about 10%, or about 15%, or about 20%, or about 30%, or about 40%, or about 50%, or about 60%, or about 70%, or about 80% higher than Epidyolex.

### Fed Conditions:

Tₘₐₓ—For each of CBD and 7-OH-CBD, about 2 to 8 hours, or about 3 to 7 hours, or about 3.5 to 5 hours, or about 4 hours. Compared to an equivalent dose of Epidyolex, the composition has a Tₘₐₓ that is about 2.5%, or about 5%, or about 10%, or about 15%, or about 20%, or about 25%, or about 30%, or about 40%, or about 50%, or about 60% faster than Epidyolex.
Cₘₐₓ—For each of CBD and 7-OH-CBD, after a dose of about 400 mg cannabidiol, a Cₘₐₓ of about 90 to 150 ng/mL, or about 100 to 145 ng/ml, or about 110 to 140 ng/ml, or about 135 ng/mL. Compared to an equivalent dose of Epidyolex, the composition has a Cₘₐₓ that is about 2.5%, or about 5%, or about 10%, or about 15%, or about 20%, or about 30%, or about 40%, or about 50%, or about 60% higher than Epidyolex.
AUC—after a dose of about 400 mg cannabidiol, a CBD AUC₀₋₂₄ of about 1500 to 2500, or about 1850 to 2250, or about 2000 to 2100, or about 2050 h*ng CBD equivalents/mL, and a 7-OH-CBD AUC₀₋₂₄ of about 400 to 700, or about 450 to 650, or about 500 to 600, or about 550 h*ng CBD equivalents/mL (the AUC₀₋₂₄ of 7-OH-CBD is multiplied by 0.9516 to obtain CBD equivalents). Compared to an equivalent dose of Epidyolex, the composition has an AUC₀₋₂₄ (sum of CBD and 7-OH-CBD) that is about 1%, or about 5%, or about 10%, or about 15%, or about 20%, or about 30%, or about 40% higher than Epidyolex.

Applicant further expects that the administration of CBD-NE under fed and fasted conditions will be safe and that the subjects do not experience significant adverse events relating to cardiovascular symptoms, respiratory rate, body temperature, blood counts, kidney and liver function, serum electrolytes, serum glucose and other serum markers.

### Additional Description of Certain Exemplary Embodiments

The specific embodiments illustrated and described herein are for illustrative purposes only, and not limiting of the invention as set forth in the appended claims.
1. A spray dried powder, comprising:
   (i) 30-45 wt.-%, based on the total weight of the spray dried powder, of cannabidiol,
   (ii) 5 to 15 wt.-%, based on the total weight of the spray dried powder, of Vitamin E,
   (iii) 2.5 to 7.5 wt.-%, based on the total weight of the spray dried powder, of tocopherol polyethylene glycol succinate,
   (iv) 25 to 40 wt.-%, based on the total weight of the spray dried powder, of gelatine,
   (v) 4 to 10 wt.-%, based on the total weight of the spray dried powder, of hydrolysed starch having a DE value selected in the range of 8 to 38,
   (vi) 0.5 to 3 wt. %, based on the total weight of the spray dried powder, of an additional antioxidant (other than Vitamin E), preferably ascorbic acid or a pharmaceutically acceptable salt thereof, and
   (vii) less than 5 wt.-%, based on the total weight of the spray dried powder, of water, wherein the spray dried powder further comprises cannabidiol hydroxyquinone in an amount of up to 0.5 wt.-%, preferably in an amount of up to 0.2 wt.-%, most preferably in an amount of up to 0.1 wt.-%, based on the total amount of the spray dried powder.
2. The spray dried powder of exemplary embodiment 1, wherein the spray dried powder has a particle size distribution measured by laser diffraction characterized by a D10 of more than 1 μm, a D50 selected in the range from 1-200 μm and a D 90 of less than 500 μm, preferably by a D10 of more than 5 μm, a D50 selected in the range from 10-150 μm and a D90 of less than 400 μm, most preferably by a D10 of more than 8 μm, a D50 selected in the range from 20-100 μm and a D90 of less than 300 μm.
3. A composition comprising the spray dried powder of exemplary embodiment 1 or 2.
4. The composition of exemplary embodiment 3, wherein the composition is a pharmaceutical composition further comprising a pharmaceutically acceptable carrier.
5. The pharmaceutical composition of exemplary embodiment 4, wherein the pharmaceutical composition, when administered in the fasted state, has a coefficient of variation (CV) of CBD below 83%, preferably below 75%, more preferably below 70%; and/or, when administered in the fed state, has a coefficient of variation (CV) of CBD below 45%.
6. The pharmaceutical composition of exemplary embodiment 4 or 5, wherein the pharmaceutical composition has a lower coefficient of variation (CV) of CBD compared to the CV of CBD provided by a liquid CBD composition, wherein the liquid CBD formulation is an alcoholic oil solution of CBD.
7. The pharmaceutical composition of any one of exemplary embodiments 4-6, for use in therapy.
8. The pharmaceutical composition of any one of exemplary embodiments 4-6, for use in therapy of a patient, or for use in the treatment of a patient in need thereof, wherein the patient can be either in the fasted or fed state.
9. The pharmaceutical composition of any one of exemplary embodiments 4-6 for use in the prevention or treatment of a patient suffering from a disease or condition selected from: chronic pain; neuropathic pain; post-operative pain; anxiety, including generalized anxiety disorder (GAD), social anxiety disorder (SAD), and post-traumatic stress disorder (PTSD); neurodegenerative diseases, including Alzheimer's and Parkinson's; autoimmune conditions, including rheumatoid arthritis and Crohn's disease; sleep disorders, including insomnia and anxiety-related sleep disturbances; diseases of the central nervous system, including multiple sclerosis, amyotrophic lateral sclerosis, Parkinson's disease, Huntington's disease, autism, and schizophrenia; opioid use disorders; Tourette's syndrome; glaucoma; IBD; IBS; and cancer and cancer related illnesses, including gliomas, glioblastomas, colorectal cancer, pancreatic cancer, lung cancer, and breast cancer.
10. The pharmaceutical composition for the use of exemplary embodiment 9, wherein the patient is either in the fasted or fed state.
11. A kit of parts for administering the pharmaceutical composition of any one of exemplary embodiments 4-6 or the pharmaceutical composition for the use of any one of exemplary embodiments 7-10 to a subject in a fed or fasted state, comprising a) the pharmaceutical composition; and b) instructions for dosing the pharmaceutical composition, wherein the instructions specify the administration of the pharmaceutical composition to the subject in a fed or fasted state.
12. A method for treating a patient suffering from a disease or condition comprising the step of orally administering to the patient the spray-dried powder of exemplary embodiment 1 or 2, the composition of exemplary embodiment 3 or 4, or the pharmaceutical composition of exemplary embodiment 5 or 6, wherein the disease or condition comprises chronic pain; neuropathic pain; post-operative pain (e.g., dental procedure or oral surgery); anxiety, including generalized anxiety disorder (GAD), social anxiety disorder (SAD), and post-traumatic stress disorder (PTSD); neurodegenerative diseases, including Alzheimer's and Parkinson's; autoimmune conditions, including rheumatoid arthritis and Crohn's disease; sleep disorders, including insomnia and anxiety-related sleep disturbances; diseases of the central nervous system, including multiple sclerosis, amyotrophic lateral sclerosis, Parkinson's disease, Huntington's disease, autism, and schizophrenia; opioid use disorders; Tourette's syndrome; glaucoma; IBD; IBS; and cancer and cancer related illnesses, including gliomas, glioblastomas, colorectal cancer, pancreatic cancer, lung cancer, and breast cancer.
13. The method of exemplary embodiment 12, wherein the step of orally administering comprises orally administering 400 mg of cannabidiol in the form of one or more capsules.
14. The method of exemplary embodiment 12 or 13, wherein the step of orally administering is performed without specifying whether the patient is in the fed or fasted state.
15. The method of any one of exemplary embodiments 12-14, wherein the step of orally administering is performed without regard to proximity to a meal.
16. A method of treating acute or chronic pain in a patient in need thereof, comprising administering to the patient a therapeutically effective dose of a pharmaceutical composition comprising:
   (i) about 20 to 50 wt.-%, based on the total weight of the composition, of cannabidiol or a pharmaceutically acceptable salt thereof;
   (ii) about 1 to 25 wt.-%, based on the total weight of the composition, of Vitamin E;
   (iii) about 1 to 15 wt.-%, based on the total weight of the composition, of a Vitamin E Derivative; and
   (iv) one or more excipients or additives in an amount to make the composition total 100 wt.-%;
      wherein the patient's pain is effectively treated without the concomitant administration of an opioid analgesic, and without regard to the proximity of a meal.
17. The method of exemplary embodiment 16, wherein the pharmaceutical composition is administered at a cannabidiol dose of about 5 mg/kg/day to 20 mg/kg/day as the sole pharmaceutical intervention to treat the pain.
18. The method of exemplary embodiment 16 or 17, wherein the composition is administered in a fasted state and achieves therapeutically effective pharmacokinetic values sufficient to treat the pain, wherein the pain is measured by the Visual Analogue Scale (VAS).
19. The method of any one of exemplary embodiments 16-18, wherein the pain is caused by a dental procedure or oral surgery, and the composition is administered to the patient under fasted conditions.
20. The method of any one of exemplary embodiments 16-19, wherein the composition is in the form of a spray-dried powder according to exemplary embodiment 1 or 2.
21. A method of treating insomnia or other sleep disorder in a patient in need thereof, comprising administering to the patient a therapeutically effective dose of a pharmaceutical composition comprising:
   (i) about 20 to 50 wt.-%, based on the total weight of the composition, of cannabidiol or a pharmaceutically acceptable salt thereof;
   (ii) about 1 to 25 wt.-%, based on the total weight of the composition, of Vitamin E;
   (iii) about 1 to 15 wt.-%, based on the total weight of the composition, of a Vitamin E Derivative; and
   (iv) one or more excipients or additives in an amount to make the composition total 100 wt.-%;
   wherein the composition is administered within about 60 minutes after a meal.
22. The method of exemplary embodiment 21, wherein the composition exhibits a Tmax at about 2 to 5 hours after administration.
23. The method of exemplary embodiment 21 or 22, wherein compared to an equivalent dose of Epidyolex, the composition has a Tmax that is about 2.5%, or about 5%, or about 10%, or about 15%, or about 20%, or about 25%, or about 30%, or about 40%, or about 50%, or about 60% faster than Epidyolex.
24. The method of any one of exemplary embodiments 21-23, wherein the meal is an evening meal and the composition is administered within 30 minutes of the meal.
25. The method of any one of exemplary embodiments 21-24, wherein the Tmax occurs at about 2 to 4 hours after administration.
26. The method of any one of exemplary embodiments 21-25, wherein the composition is in the form of a spray-dried powder according to exemplary embodiment 1 or 2.
27. The method of any one of exemplary embodiments 21-26, wherein the composition is administered at a cannabidiol dose of about 5 mg/kg/day to 20 mg/kg/day.
28. The method of any one of exemplary embodiments 21-27, wherein the composition is administered at a cannabidiol dose of about 5 mg/kg/day to 10 mg/kg/day.
29. A method of treating a disease or disorder that benefits from cannabidiol therapy in a patient in need thereof, comprising administering to the patient a therapeutically effective dose of a pharmaceutical composition comprising:
   (i) about 20 to 50 wt.-%, based on the total weight of the composition, of cannabidiol or a pharmaceutically acceptable salt thereof;
   (ii) about 1 to 25 wt.-%, based on the total weight of the composition, of Vitamin E;
   (iii) about 1 to 15 wt.-%, based on the total weight of the composition, of a Vitamin E Derivative; and
   (iv) one or more excipients or additives in an amount to make the composition total 100 wt.-%;
   wherein the composition is administered with or without food and without regard to the proximity of a meal.
30. The method of exemplary embodiment 29, wherein the composition exhibits a Tmax at about 2 to 5 hours after administration.
31. The method of exemplary embodiments 29-30, wherein the composition exhibits a Tmax sooner than a comparable dose of an alcoholic oil solution of CBD when both are administered in a fasted state.
32. The method of any one of exemplary embodiments 29-31, wherein the pharmaceutical composition has a lower coefficient of variation (CV) of CBD compared to the CV of CBD provided by a liquid CBD composition, wherein the liquid CBD formulation is an alcoholic oil solution of CBD.
33. The method of any one of exemplary embodiments 29-32, wherein the composition has a CV of CBD that is below 45% when the composition is administered in a fed state.
34. The method of exemplary embodiment 32, wherein the lower CV occurs in both a fed state and a fasted state.
35. The method of exemplary embodiment 29, wherein the pharmaceutical composition, when administered in a fasted state, has a coefficient of variation (CV) of CBD below 83%.
36. The method of exemplary embodiment 35, wherein the composition, when administered in the fasted state, has a coefficient of variation (CV) of CBD below 75%.
37. The method of exemplary embodiment 35 or 36, wherein the composition, when administered in the fasted state, has a coefficient of variation (CV) of CBD below 70%.

Those skilled in the art will appreciate that numerous changes and modifications can be made to the preferred embodiments of the invention and that such changes and modifications can be made without departing from the spirit of the invention. It is, therefore, intended that the appended exemplary embodiments cover all such equivalent variations as fall within the true spirit and scope of the invention. The disclosures of each patent, patent application, and publication cited or described in this document are hereby incorporated herein by reference, in their entirety.

1. A spray dried powder, comprising:
(i) 30-45 wt.-%, based on the total weight of the spray dried powder, of cannabidiol,
(ii) 5 to 15 wt.-%, based on the total weight of the spray dried powder, of Vitamin E,
(iii) 2.5 to 7.5 wt.-%, based on the total weight of the spray dried powder, of tocopherol polyethylene glycol succinate,
(iv) 25 to 40 wt.-%, based on the total weight of the spray dried powder, of gelatine,
(v) 4 to 10 wt.-%, based on the total weight of the spray dried powder, of hydrolysed starch having a DE value selected in the range of 8 to 38,
(vi) 0.5 to 3 wt. %, based on the total weight of the spray dried powder, of an additional antioxidant (other than Vitamin E), and
(vii) less than 5 wt.-%, based on the total weight of the spray dried powder, of water,
wherein the spray dried powder further comprises cannabidiol hydroxyquinone in an amount of up to 0.2 wt.-%, based on the total amount of the spray dried powder.

2. The spray dried powder of claim 1, wherein the additional antioxidant comprises ascorbic acid or a pharmaceutically acceptable salt thereof.

3. The spray dried powder of claim 1, wherein the spray dried powder comprises cannabidiol hydroxyquinone in an amount of up to 0.1 wt.-%, based on the total amount of the spray dried powder.

4. The spray dried powder of claim 1, wherein the spray dried powder has a particle size distribution measured by laser diffraction characterized by a D10 of more than 1 μm, a D50 selected in the range from 1-200 μm and a D 90 of less than 500 μm.

5. The spray dried powder of claim 1, wherein the spray dried powder has a particle size distribution measured by laser diffraction characterized by a D10 of more than 8 μm, a D50 selected in the range from 20-100 μm and a D90 of less than 300 μm.

6. A pharmaceutical composition a pharmaceutically acceptable carrier and a spray dried powder comprising:
(i) 30-45 wt.-%, based on the total weight of the spray dried powder, of cannabidiol,
(ii) 5 to 15 wt.-%, based on the total weight of the spray dried powder, of Vitamin E,
(iii) 2.5 to 7.5 wt.-%, based on the total weight of the spray dried powder, of tocopherol polyethylene glycol succinate,
(iv) 25 to 40 wt.-%, based on the total weight of the spray dried powder, of gelatine,
(v) 4 to 10 wt.-%, based on the total weight of the spray dried powder, of hydrolysed starch having a DE value selected in the range of 8 to 38,
(vi) 0.5 to 3 wt. %, based on the total weight of the spray dried powder, of an additional antioxidant (other than Vitamin E), and
(vii) less than 5 wt.-%, based on the total weight of the spray dried powder, of water,
wherein the spray dried powder further comprises cannabidiol hydroxyquinone in an amount of up to 0.5 wt.-%, based on the total amount of the spray dried powder.

7. The pharmaceutical composition of claim 6, wherein the pharmaceutical composition, when administered in the fasted state, has a coefficient of variation (CV) of CBD below 83%.

8. The pharmaceutical composition of claim 6, wherein the pharmaceutical composition, when administered in the fed state, has a coefficient of variation (CV) of CBD below 45%.

9. The pharmaceutical composition of claim 6, wherein the pharmaceutical composition, when administered in the fasted state, has a coefficient of variation (CV) of CBD below 83% and, when administered in the fed state, has a coefficient of variation (CV) of CBD below 45%.

10. A method for treating a patient suffering from a disease or condition comprising the step of orally administering to the patient the pharmaceutical composition of claim 6, wherein the disease or condition comprises chronic pain; neuropathic pain; post-operative pain (e.g., dental procedure or oral surgery); anxiety, including generalized anxiety disorder (GAD), social anxiety disorder (SAD), and post-traumatic stress disorder (PTSD); neurodegenerative diseases, including Alzheimer's and Parkinson's; autoimmune conditions, including rheumatoid arthritis and Crohn's disease; sleep disorders, including insomnia and anxiety-related sleep disturbances; diseases of the central nervous system, including multiple sclerosis, amyotrophic lateral sclerosis, Parkinson's disease, Huntington's disease, autism, and schizophrenia; opioid use disorders; Tourette's syndrome; glaucoma; IBD; IBS; and cancer and cancer related illnesses, including gliomas, glioblastomas, colorectal cancer, pancreatic cancer, lung cancer, and breast cancer.

11. The method of claim 10, wherein the step of orally administering comprises orally administering 400 mg of cannabidiol in the form of one or more capsules.

12. The method of claim 10, wherein the step of orally administering is performed without specifying whether the patient is in the fed or fasted state.

13. The method of claim 10, wherein the step of orally administering is performed without regard to proximity to a meal.

14. A method of treating a disease or disorder that benefits from cannabidiol therapy in a patient in need thereof, comprising administering to the patient a therapeutically effective dose of a pharmaceutical composition comprising:
(i) about 20 to 50 wt.-%, based on the total weight of the composition, of cannabidiol or a pharmaceutically acceptable salt thereof;
(ii) about 1 to 25 wt.-%, based on the total weight of the composition, of Vitamin E;
(iii) about 1 to 15 wt.-%, based on the total weight of the composition, of a Vitamin E Derivative; and
(iv) one or more excipients or additives in an amount to make the composition total 100 wt.-%;
wherein the composition is administered with or without food and without regard to the proximity of a meal.

15. The method of claim 14, wherein the composition exhibits a Tmax at about 2 to 5 hours after administration.

16. The method of claim 14, wherein the composition exhibits a Tmax sooner than a comparable dose of an alcoholic oil solution of CBD when both are administered in a fasted state.

17. The method of claim 14, wherein the pharmaceutical composition has a lower coefficient of variation (CV) of CBD compared to the CV of CBD provided by a liquid CBD composition, wherein the liquid CBD formulation is an alcoholic oil solution of CBD.

18. The method of claim 14, wherein the composition has a CV of CBD that is below 45% when the composition is administered in a fed state.

19. The method of claim 18, wherein the lower CV occurs in both a fed state and a fasted state.

20. The method of claim 14, wherein the composition, when administered in the fasted state, has a coefficient of variation (CV) of CBD below 70%.
